(19) United States
(12) Patent Application Publication
(10) Pub. No.: US 2011/0010793 A1
(43) Pub. Date: Jan. 13, 2011

(54) Methods and Compositions for Increased Yield

(76) Inventors: James Behm, Findlay, OH (US); Liesa Cerny, Chesterfield, MO (US); Thomas L. Floyd, Bloomington, IL (US); Jeffrey Hall, Normal, IL (US); David R. Wooten, Rochester, IL (US)

Correspondence Address: MONSANTO COMPANY, 800 N. LINDBERGH BLVD., ATTENTION: GAIL P. WUELLNER, IP PARALEGAL, (E1NA), ST. LOUIS, MO, 63167, US

(21) Appl. No.: 12/918,067

(22) PCT Filed: Feb. 13, 2009
(86) PCT No.: PCT/US09/33999

§ 371 Date: Aug. 18, 2010

Related U.S. Application Data
(60) Provisional application No. 61/029,585, filed on Feb. 19, 2008.

(51) Int. Cl. A01H 1/04; A01H 5/00; C07H 21/04; C12Q 1/68

(52) U.S. Cl. 800/265; 800/267; 800/312; 800/301; 800/300; 800/302; 536/23.6; 536/24.3; 435/6

(57) ABSTRACT

The invention overcomes the deficiencies of the art by providing methods for breeding soybean plants containing genomic regions associated with the pubescence alleles, T and Td, associated with increased grain yield. In addition, the invention provides the locus for Td. Moreover, the invention includes germplasm and the use of germplasm containing genomic regions conferring increased yield for introgression into elite germplasm in a breeding program. Moreover, the invention provides methods of purifying soybean breeding lines for such traits as flower color and pubescence color at early stages, such as seed. The invention also provides derivatives, and plant parts of these plants and uses thereof.

### CROSS REFERENCE TO RELATED APPLICATIONS

This application is a National Stage of International Application No. PCT/US2009/033999, filed Feb. 19, 2009, which claims the benefit of U.S. Provisional Application No. 61/029,585, filed on Feb. 19, 2008. The entire disclosures of the above applications are incorporated herein by reference.

### INCORPORATION OF SEQUENCE LISTING

A sequence listing containing the file named “pa_{—}54777.txt” which is 58,064 bytes (measured in MS-Windows) and was created on Feb. 18, 2008 comprises 130 nucleotide sequences, and is herein incorporated by reference in its entirety.

### BACKGROUND OF THE INVENTION

1. Field of the Invention

The present invention is in the field of plant breeding. More specifically, the invention includes a method for breeding soybean plants containing quantitative trait loci that are associated with pubescence and yield. The invention further includes methods and compositions of loci for screening plants from the genus Glycine with markers associated with yield. Moreover, the invention includes methods for altering flavonoid synthesis. In addition, the invention includes methods for purifying soybean breeding lines.

2. Description of Related Art

The soybean, Glycine max (L.) Merril, is a major economic crop worldwide and is a primary source of vegetable oil and protein (Sinclair and Backman, 1989). Recently, corn acreage has significantly increased as a result of the rapid growth of the corn market's ethanol sector. The main source of the additional corn acreage is from a reduction in soybean acres. However, soybean demand is expected to increase. The USDA estimated biodiesel production reached 250 million gallons in 2006, a 173-percent increase from 2005 (Anon, 2007). For the 2005/06 crop year biodiesel production accounted for 8 percent of soybean oil use; for 2006/07, biodiesel is expected to account for 2.6 billion pounds of soybean oil or 13 percent of total domestic soybean use (Anon, 2007). Therefore, an increase in soybean yield is needed to meet the needs of the market with decreasing soybean acres.

Yield is a major breeding objective due to its effect on economic return to the grower. The average rate of yield increase of soybean in the United States is estimated at 0.023 Mg ha⁻¹ yr⁻¹ (Orf et al., 2004). Yield is expressed phenotypically through morphological features and physiological functions, such as pod set and seed size. Yield is expressed genetically as a complex quantitative trait.

The narrow genetic base of soybean in North America may be impeding the rate of yield gains (Thompson et al. 1998). Six introductions, ‘Mandarin,’ ‘Manchu,’ ‘Mandarin’ (Ottawa), ‘Richland,’ ‘AK’ (Harrow), and ‘Mukden,’ contributed nearly 70% of the germplasm represented in 136 cultivar releases. This narrow genetic base is due to the small number of ancestral lines that formed the base of North American soybean germplasm, and the subsequent crossing of primarily elite lines during cultivar development.

Increasing the variability of soybean breeding populations by using parents with greater genetic diversity may lead to an increase in the rate of yield improvement (Kisha et al., 1997). Exotic germplasm has long been tapped to broaden the soybean genetic base for sustained genetic improvement (Thorne and Fehr, 1970). Guzman et al. created populations by crossing exotic germplasm (PI 68658, PI 407720, and PI 297544) with conventional breeding lines and mapped 8 quantitative trait loci (QTLs) from a PI parent using simple sequence repeat (SSR) markers (2007). Although yield QTLs have been identified in exotic germplasm, the utilization of the traits has been hampered by the presence of unfavorable genes tightly linked with the beneficial genes (Concibido et al., 2003), and by the high frequency of deleterious alleles in much of the germplasm.

Yield is closely associated with plant maturity in soybean. In addition, a number of yield QTLs mapped by Guzman et al. were associated with a delay in plant maturity (2007). An increase of one day in maturity may be equivalent to a ˜0.7 bu/A increase in yield. Conversely, a decrease in maturity is often penalized with a ˜0.7 bu/A decrease in yield. The correlation of plant maturity and yield confounds the evaluation of potential QTLs and candidate genes associated with yield. Identification of genomic regions associated with yield independent of plant maturity will assist breeders in developing varieties with increased yields.

QTLs for soybean yield have been identified in elite lines Archer, ‘Minsoy’, and ‘Noir I’ through the use of SSR marker technology (Orf et al., 1999). Archer has QTL alleles for increased yield associated with the SSR markers Satt002 (linkage group D2) and Satt144 (on linkage group F). The QTL linked to Satt002 and Satt144 accounted for 8 and 13% of the phenotypic yield variation, respectively. SSR marker analysis is a difficult process to automate. SNP marker analysis uses direct hybridization and does not require gel electrophoresis and manual gel tracking. Therefore, the process is more amenable to automation and permits for accurate and high speed detection of SNP haplotypes across thousands of individuals. In addition, SNP analysis requires less time and expense than SRR analysis.

Pubescence color may act as a phenotypic marker for yield QTLs. Soybean pubescence color may influence the microclimate of the canopy and consequently the seed yield. Lines with gray pubescence had from 7.6 to 27.7% higher yields than those with tawny pubescence in warmer years, receiving >2664 crop heat units (CHU) during the growing season (Morrison et al., 1997). Soybean lines with tawny pubescence had 9.3% higher seed yields than those with gray pubescence in cooler years receiving <2664 CHU (Morrison et al.,1997). T and Td loci control pubescence color of soybean with epistatic effects (IT TdTd, tawny; IT tdtd, light tawny or near-gray; tt TdTd or tt tdtd, gray). The T locus has been cloned and is located on C2 (Toda et al. 2002). Alleles at the T locus on linkage group C2 are associated with chilling tolerance (Toda et al., 2005). Chilling stress retards growth, causes abortion of flowers and immature pods, and reduces the final seed yield (Raper and Kramer, 1987). Furthermore, chilling temperatures (about 15° C.) during flowering induce browning and cracking of seed coats (Sunada and Ito, 1982). In contrast to the T locus, the genomic location or encoding protein of the Td locus has not been determined and has not previously been associated with factors that may influence grain yield.

There is a need in the art of plant breeding to identify QTLs associated with yield independent of soybean plant maturity. In addition, there is a need for a rapid, cost-efficient method to pre-select for yield of soybean plants. The present invention provides a method for screening and selecting a soybean plant for yield using single nucleotide polymorphism (SNP) technology.

### SUMMARY OF THE INVENTION

The present invention includes a method of introgressing an allele into a soybean plant comprising (A) crossing at least one first soybean plant comprising a nucleic acid sequence selected from the group consisting of SEQ ID NO: 1 through to SEQ ID NO: 26 with at least one second soybean plant in order to form a segregating population, (B) screening the segregating population with one or more nucleic acid markers to determine if one or more soybean plants from the segregating population contains the nucleic acid sequence, and (C) selecting from the segregation population one or more soybean plants comprising a nucleic acid sequence selected from the group consisting of SEQ ID NO: 1 to SEQ ID NO: 26. Furthermore, the invention includes a method for selecting increased yield through the use of genotypic markers associated with pubescence color.

The present invention includes a method of introgressing an allele into a soybean plant comprising: (A) crossing at soybean plant with at least one soybean plant in order to form a segregating population for pubescence color; (B) screening said segregating population with one or more nucleic acid markers to determine if one or more soybean plants from said segregating population contains a pubescence allele, wherein said pubescence allele is an allele selected from the group consisting of T and Td.

The present invention includes a soybean plant comprising a nucleic acid sequence selected from the group consisting of SEQ ID NO: 1 through SEQ ID NO: 26.

The present invention includes a substantially purified nucleic acid molecule comprising a nucleic acid sequence selected from the group consisting of SEQ ID NO: 1 through SEQ ID NO: 130 and complements thereof.

The present invention includes a soybean plant comprising a pubescence locus Td.

The present invention includes a soybean plant comprising a pubescence locus Td and T.

The present invention includes a method of purifying soybean lines for phenotypic traits comprising pubescence color and flower color at early stages, such as seed. In addition, the present invention includes methods for purifying soybean lines for phenotypic trait comprising pubescence color and flower color at early generations.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Breeding strategy to select for increased grain yield

FIG. 2A-B: Backcross breeding strategies to select for increased grain yield

### BRIEF DESCRIPTION OF NUCLEIC ACID SEQUENCES

SEQ ID NO: 1 is a genomic sequence for a polynucleotide associated with the Td locus in Glycine max (L) Merr.

SEQ ID NO: 2 is a genomic sequence for a polynucleotide associated with the Td locus in Glycine max (L) Merr.

SEQ ID NO: 3 is a genomic sequence for a polynucleotide associated with the Td locus in Glycine max (L) Merr.

SEQ ID NO: 4 is a genomic sequence for a polynucleotide associated with the Td locus in Glycine max (L) Merr.

SEQ ID NO: 5 is a genomic sequence for a polynucleotide associated with the Td locus in Glycine max (L) Merr.

SEQ ID NO: 6 is a genomic sequence for a polynucleotide associated with the Td locus in Glycine max (L) Merr.

SEQ ID NO: 7 is a genomic sequence for a polynucleotide associated with the Td locus in Glycine max (L) Merr.

SEQ ID NO: 8 is a genomic sequence for a polynucleotide associated with the Td locus in Glycine max (L) Merr.

SEQ ID NO: 9 is a genomic sequence for a polynucleotide associated with the Td locus in Glycine max (L) Merr.

SEQ ID NO: 10 is a genomic sequence for a polynucleotide associated with the Td locus in Glycine max (L) Merr.

SEQ ID NO: 11 is a genomic sequence for a polynucleotide associated with the Td locus in Glycine max (L) Merr.

SEQ ID NO: 12 is a genomic sequence for a polynucleotide associated with the Td locus in Glycine max (L) Merr.

SEQ ID NO: 13 is a genomic sequence for a polynucleotide associated with the Td locus in Glycine max (L) Merr.

SEQ ID NO: 14 is a genomic sequence for a polynucleotide associated with the Td locus in Glycine max (L) Merr.

SEQ ID NO: 15 is a genomic sequence for a polynucleotide associated with the Td locus in Glycine max (L) Merr.

SEQ ID NO: 16 is a genomic sequence for a polynucleotide associated with the Td locus in Glycine max (L) Merr.

SEQ ID NO: 17 is a genomic sequence for a polynucleotide associated with the Td locus in Glycine max (L) Merr.

SEQ ID NO: 18 is a genomic sequence for a polynucleotide associated with the T locus in Glycine max (L) Merr.

SEQ ID NO: 19 is a genomic sequence for a polynucleotide associated with the T locus in Glycine max (L) Merr.

SEQ ID NO: 20 is a genomic sequence for a polynucleotide associated with the T locus in Glycine max (L) Merr.

SEQ ID NO: 21 is a genomic sequence for a polynucleotide associated with the T locus in Glycine max (L) Merr.

SEQ ID NO: 22 is a genomic sequence for a polynucleotide associated with the T locus in Glycine max (L) Merr.

SEQ ID NO: 23 is a genomic sequence for a polynucleotide associated with the T locus in Glycine max (L) Merr.

SEQ ID NO: 24 is a genomic sequence for a polynucleotide associated with the T locus in Glycine max (L) Merr.

SEQ ID NO: 25 is a genomic sequence for a polynucleotide associated with the T locus in Glycine max (L) Merr.

SEQ ID NO: 26 is a genomic sequence for a polynucleotide associated with the T locus in Glycine max (L) Merr.

SEQ ID NO: 27 is a PCR primer for the amplification of SEQ ID NO: 1.

SEQ ID NO: 28 is a PCR primer for the amplification of SEQ ID NO: 1.

SEQ ID NO: 29 is a PCR primer for the amplification of SEQ ID NO: 2.

SEQ ID NO: 30 is a PCR primer for the amplification of SEQ ID NO: 2.

SEQ ID NO: 31 is a PCR primer for the amplification of SEQ ID NO: 3.

SEQ ID NO: 32 is a PCR primer for the amplification of SEQ ID NO: 3.

SEQ ID NO: 33 is a PCR primer for the amplification of SEQ ID NO: 4.

SEQ ID NO: 34 is a PCR primer for the amplification of SEQ ID NO: 4.

SEQ ID NO: 35 is a PCR primer for the amplification of SEQ ID NO: 5.

SEQ ID NO: 36 is a PCR primer for the amplification of SEQ ID NO: 5.

SEQ ID NO: 37 is a PCR primer for the amplification of SEQ ID NO: 6.

SEQ ID NO: 38 is a PCR primer for the amplification of SEQ ID NO: 6.

SEQ ID NO: 39 is a PCR primer for the amplification of SEQ ID NO: 7.

SEQ ID NO: 40 is a PCR primer for the amplification of SEQ ID NO: 7.

SEQ ID NO: 41 is a PCR primer for the amplification of SEQ ID NO: 8.

SEQ ID NO: 42 is a PCR primer for the amplification of SEQ ID NO: 8.

SEQ ID NO: 43 is a PCR primer for the amplification of SEQ ID NO: 9.

SEQ ID NO: 44 is a PCR primer for the amplification of SEQ ID NO: 9.

SEQ ID NO: 45 is a PCR primer for the amplification of SEQ ID NO: 10.

SEQ ID NO: 46 is a PCR primer for the amplification of SEQ ID NO: 10.

SEQ ID NO: 47 is a PCR primer for the amplification of SEQ ID NO: 11.

SEQ ID NO: 48 is a PCR primer for the amplification of SEQ ID NO: 11.

SEQ ID NO: 49 is a PCR primer for the amplification of SEQ ID NO: 12.

SEQ ID NO: 50 is a PCR primer for the amplification of SEQ ID NO: 12.

SEQ ID NO: 51 is a PCR primer for the amplification of SEQ ID NO: 13.

SEQ ID NO: 52 is a PCR primer for the amplification of SEQ ID NO: 13.

SEQ ID NO: 53 is a PCR primer for the amplification of SEQ ID NO: 14.

SEQ ID NO: 54 is a PCR primer for the amplification of SEQ ID NO: 14.

SEQ ID NO: 55 is a PCR primer for the amplification of SEQ ID NO: 15.

SEQ ID NO: 56 is a PCR primer for the amplification of SEQ ID NO: 15.

SEQ ID NO: 57 is a PCR primer for the amplification of SEQ ID NO: 16.

SEQ ID NO: 58 is a PCR primer for the amplification of SEQ ID NO: 16.

SEQ ID NO: 59 is a PCR primer for the amplification of SEQ ID NO: 17.

SEQ ID NO: 60 is a PCR primer for the amplification of SEQ ID NO: 17.

SEQ ID NO: 61 is a PCR primer for the amplification of SEQ ID NO: 18.

SEQ ID NO: 62 is a PCR primer for the amplification of SEQ ID NO: 18.

SEQ ID NO: 63 is a PCR primer for the amplification of SEQ ID NO: 19.

SEQ ID NO: 64 is a PCR primer for the amplification of SEQ ID NO: 19.

SEQ ID NO: 65 is a PCR primer for the amplification of SEQ ID NO: 20.

SEQ ID NO: 66 is a PCR primer for the amplification of SEQ ID NO: 20.

SEQ ID NO: 67 is a PCR primer for the amplification of SEQ ID NO: 21.

SEQ ID NO: 68 is a PCR primer for the amplification of SEQ ID NO: 21.

SEQ ID NO: 69 is a PCR primer for the amplification of SEQ ID NO: 22.

SEQ ID NO: 70 is a PCR primer for the amplification of SEQ ID NO: 22.

SEQ ID NO: 71 is a PCR primer for the amplification of SEQ ID NO: 23.

SEQ ID NO: 72 is a PCR primer for the amplification of SEQ ID NO: 23.

SEQ ID NO: 73 is a PCR primer for the amplification of SEQ ID NO: 24.

SEQ ID NO: 74 is a PCR primer for the amplification of SEQ ID NO: 24.

SEQ ID NO: 75 is a PCR primer for the amplification of SEQ ID NO: 25.

SEQ ID NO: 76 is a PCR primer for the amplification of SEQ ID NO: 25.

SEQ ID NO: 77 is a PCR primer for the amplification of SEQ ID NO: 26.

SEQ ID NO: 78 is a PCR primer for the amplification of SEQ ID NO: 26.

SEQ ID NO: 79 is a probe for the detection of the SNP of SEQ ID NO: 1.

SEQ ID NO: 80 is a probe for the detection of the SNP of SEQ ID NO: 1.

SEQ ID NO: 81 is a probe for the detection of the SNP of SEQ ID NO: 2.

SEQ ID NO: 82 is a probe for the detection of the SNP of SEQ ID NO: 2.

SEQ ID NO: 83 is a probe for the detection of the SNP of SEQ ID NO: 3.

SEQ ID NO: 84 is a probe for the detection of the SNP of SEQ ID NO: 3.

SEQ ID NO: 85 is a probe for the detection of the SNP of SEQ ID NO: 4.

SEQ ID NO: 86 is a probe for the detection of the SNP of SEQ ID NO: 4.

SEQ ID NO: 87 is a probe for the detection of the SNP of SEQ ID NO: 5.

SEQ ID NO: 88 is a probe for the detection of the SNP of SEQ ID NO: 5.

SEQ ID NO: 89 is a probe for the detection of the SNP of SEQ ID NO: 6.

SEQ ID NO: 90 is a probe for the detection of the SNP of SEQ ID NO: 6.

SEQ ID NO: 91 is a probe for the detection of the SNP of SEQ ID NO: 7.

SEQ ID NO: 92 is a probe for the detection of the SNP of SEQ ID NO: 7.

SEQ ID NO: 93 is a probe for the detection of the SNP of SEQ ID NO: 8.

SEQ ID NO: 94 is a probe for the detection of the SNP of SEQ ID NO: 8.

SEQ ID NO: 95 is a probe for the detection of the SNP of SEQ ID NO: 9.

SEQ ID NO: 96 is a probe for the detection of the SNP of SEQ ID NO: 9.

SEQ ID NO: 97 is a probe for the detection of the SNP of SEQ ID NO: 10.

SEQ ID NO: 98 is a probe for the detection of the SNP of SEQ ID NO: 10.

SEQ ID NO: 99 is a probe for the detection of the SNP of SEQ ID NO: 11.

SEQ ID NO: 100 is a probe for the detection of the SNP of SEQ ID NO: 11.

SEQ ID NO: 101 is a probe for the detection of the SNP of SEQ ID NO: 12.

SEQ ID NO: 102 is a probe for the detection of the SNP of SEQ ID NO: 12.

SEQ ID NO: 103 is a probe for the detection of the SNP of SEQ ID NO: 13.

SEQ ID NO: 104 is a probe for the detection of the SNP of SEQ ID NO: 13.

SEQ ID NO: 105 is a probe for the detection of the SNP of SEQ ID NO: 14.

SEQ ID NO: 106 is a probe for the detection of the SNP of SEQ ID NO: 14.

SEQ ID NO: 107 is a probe for the detection of the SNP of SEQ ID NO: 15.

SEQ ID NO: 108 is a probe for the detection of the SNP of SEQ ID NO: 15.

SEQ ID NO: 109 is a probe for the detection of the SNP of SEQ ID NO: 16.

SEQ ID NO: 110 is a probe for the detection of the SNP of SEQ ID NO: 16.

SEQ ID NO: 111 is a probe for the detection of the SNP of SEQ ID NO: 17.

SEQ ID NO: 112 is a probe for the detection of the SNP of SEQ ID NO: 17.

SEQ ID NO: 113 is a probe for the detection of the SNP of SEQ ID NO: 18.

SEQ ID NO: 114 is a probe for the detection of the SNP of SEQ ID NO: 18.

SEQ ID NO: 115 is a probe for the detection of the SNP of SEQ ID NO: 19.

SEQ ID NO: 116 is a probe for the detection of the SNP of SEQ ID NO: 19.

SEQ ID NO: 117 is a probe for the detection of the SNP of SEQ ID NO: 20.

SEQ ID NO: 118 is a probe for the detection of the SNP of SEQ ID NO: 20.

SEQ ID NO: 119 is a probe for the detection of the SNP of SEQ ID NO: 21.

SEQ ID NO: 120 is a probe for the detection of the SNP of SEQ ID NO: 21.

SEQ ID NO: 121 is a probe for the detection of the SNP of SEQ ID NO: 22.

SEQ ID NO: 122 is a probe for the detection of the SNP of SEQ ID NO: 22.

SEQ ID NO: 123 is a probe for the detection of the SNP of SEQ ID NO: 23.

SEQ ID NO: 124 is a probe for the detection of the SNP of SEQ ID NO: 23.

SEQ ID NO: 125 is a probe for the detection of the SNP of SEQ ID NO: 24.

SEQ ID NO: 126 is a probe for the detection of the SNP of SEQ ID NO: 24.

SEQ ID NO: 127 is a probe for the detection of the SNP of SEQ ID NO: 25.

SEQ ID NO: 128 is a probe for the detection of the SNP of SEQ ID NO: 25.

SEQ ID NO: 129 is a probe for the detection of the SNP of SEQ ID NO: 26.

SEQ ID NO: 130 is a probe for the detection of the SNP of SEQ ID NO: 26.

### DESCRIPTION OF ILLUSTRATIVE EMBODIMENTS

The definitions and methods provided define the present invention and guide those of ordinary skill in the art in the practice of the present invention. Unless otherwise noted, terms are to be understood according to conventional usage by those of ordinary skill in the relevant art. Definitions of common terms in molecular biology may also be found in Albert's et al., Molecular Biology of The Cell, 3^{rd} Edition, Garland Publishing, Inc.: New York, 1994; Rigger et al., Glossary of Genetics: Classical and Molecular, 5th edition, Springer-Vela: New York, 1991; and Levin, Genes V, Oxford University Press: New York, 1994. The nomenclature for DNA bases as set forth at 37 CFR §1.822 is used.

An “allele” refers to an alternative sequence at a particular locus; the length of an allele can be as small as 1 nucleotide base, but is typically larger.

A “locus” is a short sequence that is usually unique and usually found at one particular location in the genome by a point of reference; e.g., a short DNA sequence that is a gene, or part of a gene or interagency region. The loci of this invention comprise one or more polymorphisms; i.e., alternative alleles present in some individuals.

As used herein, “polymorphism” means the presence of one or more variations of a nucleic acid sequence at one or more loci in a population of one or more individuals. The variation may comprise but is not limited to one or more base changes, the insertion of one or more nucleotides or the deletion of one or more nucleotides. A polymorphism includes a single nucleotide polymorphism (SNP), a simple sequence repeat (SSR) and indels, which are insertions and deletions. A polymorphism may arise from random processes in nucleic acid replication, through mutagenesis, as a result of mobile genomic elements, from copy number variation and during the process of meiosis, such as unequal crossing over, genome duplication and chromosome breaks and fusions. The variation can be commonly found or may exist at low frequency within a population, the former having greater utility in general plant breeding and the later may be associated with rare but important phenotypic variation.

As used herein, “marker” means a polymorphic nucleic acid sequence or nucleic acid feature. A “polymorphism” is a variation among individuals in sequence, particularly in DNA sequence, or feature, such as a transcriptional profile or methylation pattern. Useful polymorphisms include single nucleotide polymorphisms (SNPs), insertions or deletions in DNA sequence (Indels), simple sequence repeats of DNA sequence (SSRs), a restriction fragment length polymorphism, a haplotype, and a tag SNP. A genetic marker, a gene, a DNA-derived sequence, a RNA-derived sequence, a promoter, a 5′ untranslated region of a gene, a 3′ untranslated region of a gene, microRNA, siRNA, a QTL, a satellite marker, a transgene, mRNA, ds mRNA, a transcriptional profile, and a methylation pattern may comprise polymorphisms. In a broader aspect, a “marker” can be a detectable characteristic that can be used to discriminate between heritable differences between organisms. Examples of such characteristics may include genetic markers, protein composition, protein levels, oil composition, oil levels, carbohydrate composition, carbohydrate levels, fatty acid composition, fatty acid levels, amino acid composition, amino acid levels, biopolymers, pharmaceuticals, starch composition, starch levels, fermentable starch, fermentation yield, fermentation efficiency, energy yield, secondary compounds, metabolites, morphological characteristics, and agronomic characteristics.

As used herein, “marker assay” means a method for detecting a polymorphism at a particular locus using a particular method, e.g. measurement of at least one phenotype (such as seed color, flower color, or other visually detectable trait), restriction fragment length polymorphism (RFLP), single base extension, electrophoresis, sequence alignment, allelic specific oligonucleotide hybridization (ASO), random amplified polymorphic DNA (RAPD), microarray-based technologies, and nucleic acid sequencing technologies, etc.

As used herein, “typing” refers to any method whereby the specific allelic form of a given soybean genomic polymorphism is determined. For example, a single nucleotide polymorphism (SNP) is typed by determining which nucleotide is present (i.e. an A, G, T, or C). Insertion/deletions (Indels) are ascertained by determining if the Indel is present. Indels can be typed by a variety of assays including, but not limited to, marker assays.

As used herein, the phrase “immediately adjacent”, when used to describe a nucleic acid molecule that hybridizes to DNA containing a polymorphism, refers to a nucleic acid that hybridizes to DNA sequences that directly abut the polymorphic nucleotide base position. For example, a nucleic acid molecule that can be used in a single base extension assay is “immediately adjacent” to the polymorphism.

As used herein, “interrogation position” refers to a physical position on a solid support that can be queried to obtain genotyping data for one or more predetermined genomic polymorphisms.

As used herein, “consensus sequence” refers to a constructed DNA sequence which identifies SNP and Indel polymorphisms in alleles at a locus. Consensus sequence can be based on either strand of DNA at the locus and states the nucleotide base of either one of each SNP in the locus and the nucleotide bases of all Indels in the locus. Thus, although a consensus sequence may not be a copy of an actual DNA sequence, a consensus sequence is useful for precisely designing primers and probes for actual polymorphisms in the locus.

As used herein, the term “single nucleotide polymorphism,” also referred to by the abbreviation “SNP,” means a polymorphism at a single site wherein said polymorphism constitutes a single base pair change, an insertion of one or more base pairs, or a deletion of one or more base pairs.

As used herein, “genotype” means the genetic component of the phenotype and it can be indirectly characterized using markers or directly characterized by nucleic acid sequencing. Suitable markers include a phenotypic character, a metabolic profile, a genetic marker, or some other type of marker. A genotype may constitute an allele for at least one genetic marker locus or a haplotype for at least one haplotype window. In some embodiments, a genotype may represent a single locus and in others it may represent a genome-wide set of loci. In another embodiment, the genotype can reflect the sequence of a portion of a chromosome, an entire chromosome, a portion of the genome, and the entire genome.

As used herein, “phenotype” means the detectable characteristics of a cell or organism which are a manifestation of gene expression.

As used herein, “linkage” refers to the relationship between two or more genes or loci that tend to be inherited together, resulting from the proximity of the loci on the chromosome.

As used herein, “linkage disequilibrium” is defined in the context of the relative frequency of gamete types in a population of many individuals in a single generation. If the frequency of allele A is p, a is p′, B is q and b is q′, then the expected frequency (with no linkage disequilibrium) of genotype AB is pq, Ab is pq′, aB is p′q and ab is p′q′. Any deviation from the expected frequency is called linkage disequilibrium. Two loci are said to be “genetically linked” when they are in linkage disequilibrium.

As used herein, “quantitative trait locus (QTL)” means a locus that controls to some degree numerically representable traits that are usually continuously distributed.

As used herein, the term “soybean” means Glycine max and includes all plant varieties that can be bred with soybean, including wild soybean species.

As used herein, the term “line” or “breeding line” refers to a group of individuals from a common ancestory.

As used herein, the term “variety” refers to a group of similar plants that by morphological features and performance can be identified from other varieties within the same species.

As used herein, the term “elite line” means any line that has resulted from breeding and selection for superior agronomic performance. An elite plant is any plant from an elite line.

As used herein, the term “flavonoid” means any phenolic compound synthesized in or following the phenylpropanoid metabolic pathway. For example, flavonoids include, but are not limited to, isoflavonoids, neoflavonoids, flavans, isoflavans, flavones, isoflavones, flavanones, isoflavanones, flavonols, hydroflavonols, biochanins, anthrocynidins, anthrocyanin and molecules derived from modification of these classes of molecules.

As used herein, the term “comprising” means “including but not limited to”.

The present invention provides plants and methods for producing plants comprising non-transgenic mutations that confer increased grain yield. Increases in yield assist growers to remain competitive with fluctuating markets. Thus, plants of the invention are of great value as to increased yields. Additionally, plants provided herein comprise agronomically elite characteristics, enabling a commercially significant yield.

### I. Plants of the Invention

The invention provides plants and derivatives thereof of soybean that combine non-transgenic traits conferring increased grain yield. In certain embodiments, the increase in grain of plants of the invention may be at least 0.5, 1, 1.5, 2.0, 2.5, or 3 bushels/acre. One aspect of the current invention is therefore directed to the aforementioned plants and parts thereof and methods for using these plants and plant parts. Plant parts include, but are not limited to, pollen, an ovule and a cell. The invention further provides tissue cultures of regenerable cells of these plants, which cultures regenerate soybean plants capable of expressing all the physiological and morphological characteristics of the starting variety. Such regenerable cells may include embryos, meristematic cells, pollen, leaves, roots, root tips or flowers, or protoplasts or callus derived therefrom. Also provided by the invention are soybean plants regenerated from such a tissue culture, wherein the plants are capable of expressing all the physiological and morphological characteristics of the starting plant variety from which the regenerable cells were obtained.

### II. Marker Assisted Selection for Production of Soybean Varieties with Non-Transgenic Alleles that Confer an Increased Grain Yield

The present invention describes methods to produce soybean plants with increased grain yield. Moreover, the invention provides genetic markers and methods for the introduction of non-transgenic alleles that confer an increased grain yield. Certain aspects of the invention also provide methods for selecting parents for breeding of plants with increased grain yield. One method involves screening germplasm for pubescence color of the plant. Another method of the invention allows the creation of plants that combine alleles that confer increases in grain yield. Using the methods of the invention, loci conferring increased grain yield may be introduced into a desired soybean genetic background, for example, in the production of new commercial varieties with increased grain yield.

Marker assisted introgression involves the transfer of a chromosome region defined by one or more markers from one germplasm to a second germplasm. The initial step in that process is the localization of the trait by gene mapping, which is the process of determining the position of a gene relative to other genes and genetic markers through linkage analysis. The basic principle for linkage mapping is that the closer together two genes are on the chromosome, the more likely they are to be inherited together. Briefly, a cross is generally made between two genetically compatible but divergent parents relative to traits under study. Genetic markers can then be used to follow the segregation of traits under study in the progeny from the cross, often a backcross (BC1), F₂, or recombinant inbred population.

The term quantitative trait loci, or QTL, is used to describe regions of a genome showing quantitative or additive effects upon a phenotype. The yield loci represent exemplary QTL since multiple yield alleles result in increasing grain yield. Herein identified are genetic markers for non-transgenic yield alleles that enable breeding of soybean plants comprising the non-transgenic, yield alleles with agronomically superior plants, and selection of progeny that inherited the yield alleles. Thus, the invention allows the use of molecular tools to combine these QTLs with desired agronomic characteristics.

A. Development and Use of Linked Genetic Markers

A sample first plant population may be genotyped for an inherited genetic marker to form a genotypic database. As used herein, an “inherited genetic marker” is an allele at a single locus. A locus is a position on a chromosome, and allele refers to conditions of genes; that is, different nucleotide sequences, at those loci. The marker allelic composition of each locus can be either homozygous or heterozygous. In order for information to be gained from a genetic marker in a cross, the marker must be polymorphic; that is, it must exist in different forms so that the chromosome carrying the mutant gene can be distinguished from the chromosome with the normal gene by the form of the marker it also carries.

Formation of a phenotypic database can be accomplished by making direct observations of one or more traits on progeny derived from artificial or natural self-pollination of a sample plant or by quantitatively assessing the combining ability of a sample plant. By way of example, a plant line may be crossed to, or by, one or more testers. Testers can be inbred lines, single, double, or multiple cross hybrids, or any other assemblage of plants produced or maintained by controlled or free mating, or any combination thereof. For some self-pollinating plants, direct evaluation without progeny testing is preferred.

To map a particular trait by the linkage approach, it is necessary to establish a positive correlation in inheritance of a specific chromosomal locus with the inheritance of the trait. In the case of complex inheritance, such as with quantitative traits, linkage will generally be much more difficult to discern. In this case, statistical procedures may be needed to establish the correlation between phenotype and genotype. This may further necessitate examination of many offspring from a particular cross, as individual loci may have small contributions to an overall phenotype.

Coinheritance, or genetic linkage, of a particular trait and a marker suggests that they are physically close together on the chromosome. Linkage is determined by analyzing the pattern of inheritance of a gene and a marker in a cross. The unit of genetic map distance is the centimorgan (cM), which increases with increasing recombination. Two markers are one centimorgan apart if they recombine in meiosis about once in every 100 opportunities that they have to do so. The centimorgan is a genetic measure, not a physical one. In particular embodiments of the invention, a marker used may be defined as located less than about 45, 35, 25, 15, 10, 5, 4, 3, 2, or 1 or less cM apart from a locus.

During meiosis, pairs of homologous chromosomes come together and exchange segments in a process called recombination. The further a marker is from a gene, the more chance there is that there will be recombination between the gene and the marker. In a linkage analysis, the coinheritance of marker and gene or trait are followed in a particular cross. The probability that their observed inheritance pattern could occur by chance alone, i.e., that they are completely unlinked, is calculated. The calculation is then repeated assuming a particular degree of linkage, and the ratio of the two probabilities (no linkage versus a specified degree of linkage) is determined. This ratio expresses the odds for (and against) that degree of linkage, and because the logarithm of the ratio is used, it is known as the logarithm of the odds, e.g. a lod score. A lod score equal to or greater than 3, for example, is taken to confirm that a marker is linked to a QTL for the trait of interest. This represents 1000:1 odds that the two loci are linked Calculations of linkage are greatly facilitated by use of statistical analysis employing programs.

The genetic linkage of marker molecules to putative QTL can be established by a gene mapping model such as, without limitation, the flanking marker model reported by Lander and Botstein (1989), and interval mapping, based on maximum likelihood methods described by Lander and Botstein (1989), and implemented in the software package MAPMAKER/QTL. Additional software includes Qgene, Version 2.23 (1996) (Department of Plant Breeding and Biometry, 266 Emerson Hall, Cornell University, Ithaca, N.Y.) and Windows QTL Catagrapher 2.5 (2006) (Program in Statistitical Genetics, NC State University, Raleigh N.C.).

B. Inherited Markers

Genetic markers comprise detected differences (polymorphisms) in the genetic information carried by two or more plants. Genetic mapping of a locus with genetic markers typically requires two fundamental components: detectably polymorphic alleles and recombination or segregation of those alleles. In plants, the recombination measured is virtually always meiotic, and therefore, the two inherent requirements of plant gene mapping are polymorphic genetic markers and one or more plants in which those alleles are segregating.

Markers are preferably inherited in codominant fashion so that the presence of both alleles at a diploid locus is readily detectable, and they are free of environmental variation, i.e., their heritability is 1. A marker genotype typically comprises two marker alleles at each locus in a diploid organism such as soybeans. The marker allelic composition of each locus can be either homozygous or heterozygous. Homozygosity is a condition where both alleles at a locus are characterized by the same nucleotide sequence. Heterozygosity refers to different conditions of the gene at a locus.

A number of different marker types are available for use in genetic mapping. Exemplary genetic marker types for use with the invention include, but are not limited to, restriction fragment length polymorphisms (RFLPs), simple sequence length polymorphisms (SSLPs), amplified fragment length polymorphisms (AFLPs), single nucleotide polymorphisms (SNPs), nucleotide insertions and/or deletions (INDELs) and isozymes. Polymorphisms comprising as little as a single nucleotide change can be assayed in a number of ways. For example, detection can be made by electrophoretic techniques including a single strand conformational polymorphism (Orita et al., 1989), denaturing gradient gel electrophoresis (Myers et al., 1985), or cleavage fragment length polymorphisms (Life Technologies, Inc., Gathersberg, Md. 20877), but the widespread availability of DNA sequencing machines often makes it easier to just sequence amplified products directly. Once the polymorphic sequence difference is known, rapid assays can be designed for progeny testing, typically involving some version of PCR amplification of specific alleles (PASA, Sommer, et al., 1992), or PCR amplification of multiple specific alleles (PAMSA, Dutton and Sommer, 1991). The analysis may be used to select for genes, QTL, alleles, or genomic regions (haplotypes) that comprise or are linked to a genetic marker.

Nucleic acid analysis methods are known in the art and include, but are not limited to, PCR-based detection methods (for example, TaqMan assays), microarray methods, and nucleic acid sequencing methods. The detection of polymorphic sites in a sample of DNA, RNA, or cDNA may be facilitated through the use of nucleic acid amplification methods.

One method for detection of SNPs in DNA, RNA and cDNA samples is by use of PCR in combination with fluorescent probes for the polymorphism, as described in Livak et al., 1995 and U.S. Pat. No. 5,604,099, incorporated herein by reference. Such methods specifically increase the concentration of polynucleotides that span the polymorphic site, or include that site and sequences located either distal or proximal to it. Such amplified molecules can be readily detected by gel electrophoresis, fluorescence detection methods, or other means. Briefly, probe oligonucleotides, one of which anneals to the SNP site and the other which anneals to the wild type sequence, are synthesized. It is preferable that the site of the SNP be near the 5′ terminus of the probe oligonucleotides. Each probe is then labeled on the 3′ end with a non-fluorescent quencher and a minor groove binding moiety which lower background fluorescence and lower the Tₘ of the oligonucleotide, respectively. The 5′ ends of each probe are labeled with a different fluorescent dye wherein fluorescence is dependent upon the dye being cleaved from the probe. Some non-limiting examples of such dyes include VIC™ and 6-FAM™. DNA suspected of comprising a given SNP is then subjected to PCR using a polymerase with 5′-3′ exonuclease activity and flanking primers. PCR is performed in the presence of both probe oligonucleotides. If the probe is bound to a complimentary sequence in the test DNA then exonuclease activity of the polymerase releases a fluorescent label activating its fluorescent activity. Therefore, test DNA that contains only a wild type sequence will exhibit fluorescence associated with the label on the wild type probe. On the other hand, DNA containing only the SNP sequence will have fluorescent activity from the label on the SNP probe. However, when the DNA is from heterogeneous sources, significant fluorescence of both labels will be observed. This type of indirect genotyping at known SNP sites enables inexpensive high throughput screening of DNA samples. Thus, such a system is ideal for the identification of progeny soybean plants comprising α-subunit alleles.

Restriction fragment length polymorphisms (RFLPs) are genetic differences detectable by DNA fragment lengths, typically revealed by agarose gel electrophoresis after restriction endonuclease digestion of DNA. There are large numbers of restriction endonucleases available, characterized by their nucleotide cleavage sites and their source, e.g., EcoRI. RFLPs result from both single-bp polymorphisms within restriction site sequences and measurable insertions or deletions within a given restriction fragment. RFLPs are easy and relatively inexpensive to generate (require a cloned DNA, but no sequence) and are co-dominant. RFLPs have the disadvantage of being labor-intensive in the typing stage, although this can be alleviated to some extent by multiplexing many of the tasks and re-utilization of blots. Most RFLP are biallelic and of lesser polymorphic content than microsatellites. For these reasons, the use of RFLP in plant genetic maps has waned.

One skilled in the art would recognize that many types of molecular markers are useful as tools to monitor genetic inheritance and are not limited to RFLPs, SSRs and SNPs, and one of skill would also understand that a variety of detection methods may be employed to track various molecular markers. One skilled in the art would also recognize that markers of different types may be used for mapping, especially as technology evolves and new types of markers and means for identification are developed.

For purposes of convenience, inherited marker genotypes may be converted to numerical scores, e.g., if there are 2 forms of a SNP, or other marker, designated A and B, at a particular locus using a particular enzyme, then diploid complements may be converted to a numerical score, for example, are AA=2, AB=1, and BB=0; or AA=1, AB=0 and BB=−1. The absolute values of the scores are not important. What is important is the additive nature of the numeric designations. The above scores relate to codominant markers. A similar scoring system can be given that is consistent with dominant markers.

C. Marker Assisted Selection

The invention provides soybean plants with increased grain yield and agronomically elite characteristics. Such plants may be produced in accordance with the invention by marker assisted selection methods comprising assaying genomic DNA for the presence of markers that are genetically linked to the T and Td allele, including all possible combinations thereof.

In certain embodiments of the invention, it may be desired to obtain additional markers linked to yield alleles. This may be carried out, for example, by first preparing an F₂ population by selfing an F₁ hybrid produced by crossing inbred varieties only one of which comprises a yield allele. Recombinant inbred lines (RIL) (genetically related lines; developed from selfing F₂ lines towards homozygosity) can then be prepared and used as a mapping population. Information obtained from dominant markers can be maximized by using RIL because all loci are homozygous or nearly so.

Backcross populations [e.g., generated from a cross between a desirable variety (recurrent parent) and another variety (donor parent)] carrying a trait not present in the former can also be utilized as a mapping population. A series of backcrosses to the recurrent parent can be made to recover most of its desirable traits. Thus, a population is created consisting of individuals similar to the recurrent parent but each individual carries varying amounts of genomic regions from the donor parent. Backcross populations can be useful for mapping dominant markers if all loci in the recurrent parent are homozygous and the donor and recurrent parent have contrasting polymorphic marker alleles (Reiter et al., 1992).

Near-isogenic line (NIL) are useful for mapping purposes. NILs may be created by many backcrosses to produce an array of individuals that are nearly identical in genetic composition except for the desired trait or genomic region can be used as a mapping population. Preferably, NILs can be developed by selfing a relatively inbred individual that is still heterozygous at the genomic region or trait of interest. In mapping with NILs, only a portion of the polymorphic loci are expected to map to a selected region. Mapping may also be carried out on transformed plant lines.

D. Plant Breeding Methods

Certain aspects of the invention provide methods for marker assisted breeding of plants that enable the introduction of non-transgenic yield alleles into a heterologous soybean genetic background. In general, breeding techniques take advantage of a plant's method of pollination. There are two general methods of pollination: self-pollination which occurs if pollen from one flower is transferred to the same or another flower of the same plant, and cross-pollination which occurs if pollen comes to it from a flower on a different plant. Plants that have been self-pollinated and selected for type over many generations become homozygous at almost all gene loci and produce a uniform population of true breeding, homozygous plants.

Pedigree breeding may be used in development of suitable varieties. The pedigree breeding method for specific traits involves crossing two genotypes. Each genotype can have one or more desirable characteristics lacking in the other or each genotype can complement the other. If the two original parental genotypes do not provide all of the desired characteristics, other genotypes can be included in the breeding population. Two parents which possess favorable, complementary traits are crossed to produce an F₁. An F₂ population is produced by selfing one or several F₁'s. Selection of the best individuals may begin in the F₂ population (or later depending upon the breeder's objectives); then, beginning in the F₃ generation, the best individuals in the best families can be selected. Replicated testing of families can begin in the F₃ or F₄ generation to improve the effectiveness of selection for traits with low heritability. At an advanced stage of inbreeding (i.e., F₆ and F₇), the best lines or mixtures of phenotypically similar lines are tested for potential release as new varieties.

Each breeding program should include a periodic, objective evaluation of the efficiency of the breeding procedure. Evaluation criteria vary depending on the goal and objectives. Promising advanced breeding lines are thoroughly tested and compared to appropriate standards in environments representative of the commercial target area(s) for generally three or more years. Identification of individuals that are genetically superior is difficult because genotypic value can be masked by confounding plant traits or environmental factors. One method of identifying a superior plant is to observe its performance relative to other experimental plants and to one or more widely grown standard varieties. Single observations can be inconclusive, while replicated observations provide a better estimate of genetic worth.

Mass and recurrent selections can be used to improve populations of either self-or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued. Descriptions of other breeding methods that are commonly used for different traits and crops can be found in one of several reference books (e.g., Allard, 1960; Simmonds, 1979; Sneep et al., 1979; Fehr, 1987a,b).

The effectiveness of selecting for genotypes with traits of interest (e.g., high yield, disease resistance, fatty acid profile) in a breeding program will depend upon: 1) the extent to which the variability in the traits of interest of individual plants in a population is the result of genetic factors and is thus transmitted to the progenies of the selected genotypes; and 2) how much the variability in the traits of interest among the plants is due to the environment in which the different genotypes are growing. The inheritance of traits ranges from control by one major gene whose expression is not influenced by the environment (i.e., qualitative characters) to control by many genes whose effects are greatly influenced by the environment (i.e., quantitative characters). Breeding for quantitative traits such as yield is further characterized by the fact that: 1) the differences resulting from the effect of each gene are small, making it difficult or impossible to identify them individually; 2) the number of genes contributing to a character is large, so that distinct segregation ratios are seldom if ever obtained; and 3) the effects of the genes may be expressed in different ways based on environmental variation. Therefore, the accurate identification of transgressive segregates or superior genotypes with the traits of interest is extremely difficult and its success is dependent on the plant breeder's ability to minimize the environmental variation affecting the expression of upon quantitative character in the population.

The likelihood of identifying a transgressive segregant is greatly reduced as the number of traits combined into one genotype is increased. For example, if a cross is made between cultivars differing in three complex characters, such as yield, disease resistance and at least a first agronomic trait, it is extremely difficult without molecular tools to recover simultaneously by recombination the maximum number of favorable genes for each of the three characters into one genotype. Consequently, all the breeder can generally hope for is to obtain a favorable assortment of genes for the first complex character combined with a favorable assortment of genes for the second character into one genotype in addition to a selected gene.

Backcrossing is an efficient method for transferring specific desirable traits. This can be accomplished, for example, by first crossing a superior variety inbred (A) (recurrent parent) to a donor inbred (non-recurrent parent), which carries the appropriate gene(s) for the trait in question (Fehr, 1987). The progeny of this cross are then mated back to the superior recurrent parent (A) followed by selection in the resultant progeny for the desired trait to be transferred from the non-recurrent parent. Such selection can be based on genetic assays, as mentioned below, or alternatively, can be based on the phenotype of the progeny plant. After five or more backcross generations with selection for the desired trait, the progeny are heterozygous for loci controlling the characteristic being transferred, but are like the superior parent for most or almost all other genes. The last generation of the backcross is selfed, or sibbed, to give pure breeding progeny for the gene(s) being transferred, for example, loci providing the plant with decreased seed glycinin content.

In one embodiment of the invention, the process of backcross conversion may be defined as a process including the steps of:
(a) crossing a plant of a first genotype containing one or more desired gene, DNA sequence or element, such as T allele and Td allele associated with increase in grain yield, to a plant of a second genotype lacking said desired gene, DNA sequence or element;
(b) selecting one or more progeny plant(s) containing the desired gene, DNA sequence or element;
(c) crossing the progeny plant to a plant of the second genotype; and
(d) repeating steps (b) and (c) for the purpose of transferring said desired gene, DNA sequence or element from a plant of a first genotype to a plant of a second genotype.

Introgression of a particular DNA element or set of elements into a plant genotype is defined as the result of the process of backcross conversion. A plant genotype into which a DNA sequence has been introgressed may be referred to as a backcross converted genotype, line, inbred, or hybrid. Similarly a plant genotype lacking the desired DNA sequence may be referred to as an unconverted genotype, line, inbred, or hybrid. During breeding, the genetic markers linked to increased grain yield may be used to assist in breeding for the purpose of producing soybean plants with increased grain yield. Backcrossing and marker assisted selection in particular can be used with the present invention to introduce the increased grain yield in accordance with the current invention into any variety.

The selection of a suitable recurrent parent is an important step for a successful backcrossing procedure. The goal of a backcross protocol is to alter or substitute a trait or characteristic in the original inbred. To accomplish this, one or more loci of the recurrent inbred is modified or substituted with the desired gene from the nonrecurrent parent, while retaining essentially all of the rest of the desired genetic, and therefore the desired physiological and morphological, constitution of the original inbred. The choice of the particular nonrecurrent parent will depend on the purpose of the backcross, which in the case of the present invention may be to add one or more allele(s) conferring increased yield content. The exact backcrossing protocol will depend on the characteristic or trait being altered to determine an appropriate testing protocol. Although backcrossing methods are simplified when the characteristic being transferred is a dominant allele, a recessive allele may also be transferred. In this instance it may be necessary to introduce a test of the progeny to determine if the desired characteristic has been successfully transferred. In the case of the present invention, one may test the grain yield of progeny lines generated during the backcrossing program, as well as using the marker system described herein to select lines based upon markers rather than visual traits.

Soybean plants (Glycine max L.) can be crossed by either natural or mechanical techniques (see, e.g., Fehr, 1980). Natural pollination occurs in soybeans either by self pollination or natural cross pollination, which typically is aided by pollinating organisms. In either natural or artificial crosses, flowering and flowering time are an important consideration. Soybean is a short-day plant, but there is considerable genetic variation for sensitivity to photoperiod (Hamner, 1969; Criswell and Hume, 1972). The critical day length for flowering ranges from about 13 h for genotypes adapted to tropical latitudes to 24 h for photoperiod-insensitive genotypes grown at higher latitudes (Shibles et al., 1975). Soybeans seem to be insensitive to day length for 9 days after emergence. Photoperiods shorter than the critical day length are required for 7 to 26 days to complete flower induction (Borthwick and Parker, 1938; Shanmugasundaram and Tsou, 1978).

Either with or without emasculation of the female flower, hand pollination can be carried out by removing the stamens and pistil with a forceps from a flower of the male parent and gently brushing the anthers against the stigma of the female flower. Access to the stamens can be achieved by removing the front sepal and keel petals, or piercing the keel with closed forceps and allowing them to open to push the petals away. Brushing the anthers on the stigma causes them to rupture, and the highest percentage of successful crosses is obtained when pollen is clearly visible on the stigma. Pollen shed can be checked by tapping the anthers before brushing the stigma. Several male flowers may have to be used to obtain suitable pollen shed when conditions are unfavorable, or the same male may be used to pollinate several flowers with good pollen shed.

Genetic male sterility is available in soybeans and may be useful to facilitate hybridization in the context of the current invention, particularly for recurrent selection programs (Brim and Stuber, 1973). The distance required for complete isolation of a crossing block is not clear; however, outcrossing is less than 0.5% when male-sterile plants are 12 m or more from a foreign pollen source (Boerma and Moradshahi, 1975). Plants on the boundaries of a crossing block probably sustain the most outcrossing with foreign pollen and can be eliminated at harvest to minimize contamination.

Once harvested, pods are typically air-dried at not more than 38° C. until the seeds contain 13% moisture or less, then the seeds are removed by hand. Seed can be stored satisfactorily at about 25° C. for up to a year if relative humidity is 50% or less. In humid climates, germination percentage declines rapidly unless the seed is dried to 7% moisture and stored in an air-tight container at room temperature. Long-term storage in any climate is best accomplished by drying seed to 7% moisture and storing it at 10° C. or less in a room maintained at 50% relative humidity and in an air-tight container.

### III. Traits for Modification and Improvement of Soybean Varieties

In certain embodiments, a soybean plant provided by the invention may comprise one or more transgene(s). One example of such a transgene confers herbicide resistance. Common herbicide resistance genes include an EPSPS gene conferring glyphosate resistance, a neomycin phosphotransferase II (nptII) gene conferring resistance to kanamycin (Fraley et al., 1983), a hygromycin phosphotransferase gene conferring resistance to the antibiotic hygromycin (Vanden Elzen et al., 1985), genes conferring resistance to glufosinate or broxynil (Comai et al., 1985; Gordon-Kamm et al., 1990; Stalker et al., 1988) such as dihydrofolate reductase and acetolactate synthase (Eichholtz et al., 1987, Shah et al., 1986, Charest et al., 1990). Further examples include mutant ALS and AHAS enzymes conferring resistance to imidazalinone or a sulfonylurea (Lee et al., 1988; Miki et al., 1990), a phosphinothricin-acetyl-transferase gene conferring phosphinothricin resistance (European Appln. 0 242 246), genes conferring resistance to phenoxy proprionic acids and cycloshexones, such as sethoxydim and haloxyfop (Marshall et al., 1992); and genes conferring resistance to triazine (psbA and gs+ genes) and benzonitrile (nitrilase gene) (Przibila et al., 1991).

A plant of the invention may also comprise a gene that confers resistance to insect, pest, viral or bacterial attack. For example, a gene conferring resistance to a pest, such as soybean cyst nematode was described in PCT Application WO96/30517 and PCT Application WO93/19181. Jones et al., (1994) describe cloning of the tomato Cf-9 gene for resistance to Cladosporium fulvum); Martin et al., (1993) describe a tomato Pto gene for resistance to Pseudomonas syringae pv. and Mindrinos et al., (1994) describe an Arabidopsis RSP2 gene for resistance to Pseudomonas syringae. Bacillus thuringiensis endotoxins may also be used for insect resistance. (See, for example, Geiser et al., (1986). A vitamin-binding protein such as avidin may also be used as a larvicide (PCT application US93/06487).

The use of viral coat proteins in transformed plant cells is known to impart resistance to viral infection and/or disease development affected by the virus from which the coat protein gene is derived, as well as by related viruses. (See Beachy et al., 1990). Coat protein-mediated resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus. Developmental-arrestive proteins produced in nature by a pathogen or a parasite may also be used. For example, Logemann et al., (1992), have shown that transgenic plants expressing the barley ribosome-inactivating gene have an increased resistance to fungal disease.

Transgenes conferring increased nutritional value or another value-added trait may also be used. One example is modified fatty acid metabolism achieved by transforming a plant with an antisense gene of stearoyl-ACP desaturase to increase stearic acid content of the plant. (See Knutzon et al., 1992). A sense desaturase gene may also be introduced to alter fatty acid content. Phytate content may be modified by introduction of a phytase-encoding gene to enhance breakdown of phytate, adding more free phosphate to the transformed plant. Modified carbohydrate composition may also be affected, for example, by transforming plants with a gene coding for an enzyme that alters the branching pattern of starch. (See Shiroza et al., 1988) (nucleotide sequence of Streptococcus mutans fructosyltransferase gene); Steinmetz et al., (1985) (nucleotide sequence of Bacillus subtilis levansucrase gene); Pen et al., (1992) (production of transgenic plants that express Bacillus licheniformis α-amylase); Elliot et al., (1993) (nucleotide sequences of tomato invertase genes); Søgaard et al., (1993) (site-directed mutagenesis of barley a-amylase gene); and Fisher et al., (1993) (maize endosperm starch branching enzyme II).

Transgenes may also be used to alter protein metabolism. For example, U.S. Pat. No. 5,545,545 describes lysine-insensitive maize dihydrodipicolinic acid synthase (DHPS), which is substantially resistant to concentrations of L-lysine which otherwise inhibit the activity of native DHPS. Similarly, EP 0640141 describes sequences encoding lysine-insensitive aspartokinase (AK) capable of causing a higher than normal production of threonine, as well as a subfragment encoding antisense lysine ketoglutarate reductase for increasing lysine.

In another embodiment, a transgene may be employed that alters plant carbohydrate metabolism. For example, fructokinase genes are known for use in metabolic engineering of fructokinase gene expression in transgenic plants and their fruit (see U.S. Pat. No. 6,031,154). A further example of transgenes that may be used are genes that alter grain yield. For example, U.S. Pat. No. 6,486,383 describes modification of starch content in plants with subunit proteins of adenosine diphosphoglucose pyrophosphorylase (“ADPG PPase”). In EP0797673, transgenic plants are discussed in which the introduction and expression of particular DNA molecules results in the formation of easily mobilized phosphate pools outside the vacuole and an enhanced biomass production and/or altered flowering behavior. Still further known are genes for altering plant maturity. U.S. Pat. No. 6,774,284 describes DNA encoding a plant lipase and methods of use thereof for controlling senescence in plants. U.S. Pat. No. 6,140,085 discusses FCA genes for altering flowering characteristics, particularly timing of flowering. U.S. Pat. No. 5,637,785 discusses genetically modified plants having modulated flower development such as having early floral meristem development and comprising a structural gene encoding the LEAFY protein in its genome.

Genes for altering plant morphological characteristics are also known and may be used in accordance with the invention. U.S. Pat. No. 6,184,440 discusses genetically engineered plants which display altered structure or morphology as a result of expressing a cell wall modulation transgene. Examples of cell wall modulation transgenes include a cellulose binding domain, a cellulose binding protein, or a cell wall modifying protein or enzyme such as endoxyloglucan transferase, xyloglucan endo-transglycosylase, an expansin, cellulose synthase, or a novel isolated endo-1,4-β-glucanase.

Methods for introduction of a transgene are well known in the art and include biological and physical plant transformation protocols. See, for example, Mild et al. (1993).

Once a transgene is introduced into a variety it may readily be transferred by crossing. By using backcrossing, essentially all of the desired morphological and physiological characteristics of a variety are recovered in addition to the locus transferred into the variety via the backcrossing technique. Backcrossing methods can be used with the present invention to improve or introduce a characteristic into a plant (Poehlman et al., 1995; Fehr, 1987a,b).

### IV. Tissue Cultures and in vitro Regeneration of Soybean Plants

A further aspect of the invention relates to tissue cultures of a soybean variety of the invention. As used herein, the term “tissue culture” indicates a composition comprising isolated cells of the same or a different type or a collection of such cells organized into parts of a plant. Exemplary types of tissue cultures are protoplasts, calli and plant cells that are intact in plants or parts of plants, such as embryos, pollen, flowers, leaves, roots, root tips, anthers, and the like. In a preferred embodiment, the tissue culture comprises embryos, protoplasts, meristematic cells, pollen, leaves or anthers.

Exemplary procedures for preparing tissue cultures of regenerable soybean cells and regenerating soybean plants therefrom, are disclosed in U.S. Pat. No. 4,992,375; U.S. Pat. No. 5,015,580; U.S. Pat. No. 5,024,944, and U.S. Pat. No. 5,416,011, each of the disclosures of which is specifically incorporated herein by reference in its entirety.

An important ability of a tissue culture is the capability to regenerate fertile plants. This allows, for example, transformation of the tissue culture cells followed by regeneration of transgenic plants. For transformation to be efficient and successful, DNA must be introduced into cells that give rise to plants or germ-line tissue.

Soybeans typically are regenerated via two distinct processes; shoot morphogenesis and somatic embryogenesis (Finer, 1996). Shoot morphogenesis is the process of shoot meristem organization and development. Shoots grow out from a source tissue and are excised and rooted to obtain an intact plant. During somatic embryogenesis, an embryo (similar to the zygotic embryo), containing both shoot and root axes, is formed from somatic plant tissue. An intact plant rather than a rooted shoot results from the germination of the somatic embryo.

Shoot morphogenesis and somatic embryogenesis are different processes and the specific route of regeneration is primarily dependent on the explant source and media used for tissue culture manipulations. While the systems are different, both systems show variety-specific responses where some lines are more responsive to tissue culture manipulations than others. A line that is highly responsive in shoot morphogenesis may not generate many somatic embryos. Lines that produce large numbers of embryos during an ‘induction’ step may not give rise to rapidly-growing proliferative cultures. Therefore, it may be desired to optimize tissue culture conditions for each soybean line. These optimizations may readily be carried out by one of skill in the art of tissue culture through small-scale culture studies. In addition to line-specific responses, proliferative cultures can be observed with both shoot morphogenesis and somatic embryogenesis. Proliferation is beneficial for both systems, as it allows a single, transformed cell to multiply to the point that it will contribute to germ-line tissue.

Shoot morphogenesis was first reported by Wright et al. (1986) as a system whereby shoots were obtained de novo from cotyledonary nodes of soybean seedlings. The shoot meristems were formed subepidermally and morphogenic tissue could proliferate on a medium containing benzyl adenine (BA). This system can be used for transformation if the subepidermal, multicellular origin of the shoots is recognized and proliferative cultures are utilized. The idea is to target tissue that will give rise to new shoots and proliferate those cells within the meristematic tissue to lessen problems associated with chimerism. Formation of chimeras, resulting from transformation of only a single cell in a meristem, are problematic if the transformed cell is not adequately proliferated and does not give rise to germ-line tissue. Once the system is well understood and reproduced satisfactorily, it can be used as one target tissue for soybean transformation.

Somatic embryogenesis in soybean was first reported by Christianson et al. (1983) as a system in which embryogenic tissue was initially obtained from the zygotic embryo axis. These embryogenic cultures were proliferative but the repeatability of the system was low and the origin of the embryos was not reported. Later histological studies of a different proliferative embryogenic soybean culture showed that proliferative embryos were of apical or surface origin with a small number of cells contributing to embryo formation. The origin of primary embryos (the first embryos derived from the initial explant) is dependent on the explant tissue and the auxin levels in the induction medium (Hartweck et al., 1988). With proliferative embryonic cultures, single cells or small groups of surface cells of the ‘older’ somatic embryos form the ‘newer’ embryos.

Embryogenic cultures can also be used successfully for regeneration, including regeneration of transgenic plants, if the origin of the embryos is recognized and the biological limitations of proliferative embryogenic cultures are understood. Biological limitations include the difficulty in developing proliferative embryogenic cultures and reduced fertility problems (culture-induced variation) associated with plants regenerated from long-term proliferative embryogenic cultures. Some of these problems are accentuated in prolonged cultures. The use of more recently cultured cells may decrease or eliminate such problems.

### V. Utilization of Soybean Plants

A soybean plant provided by the invention may be used for any purpose deemed of value. Common uses include the preparation of food for human consumption, feed for non-human animal consumption and industrial uses. As used herein, “industrial use” or “industrial usage” refers to non-food and non-feed uses for soybeans or soy-based products.

Soybeans are commonly processed into two primary products, soybean protein (meal) and crude soybean oil. Both of these products are commonly further refined for particular uses. Refined oil products can be broken down into glycerol, fatty acids and sterols. These can be for food, feed or industrial usage. Edible food product use examples include coffee creamers, margarine, mayonnaise, pharmaceuticals, salad dressings, shortenings, bakery products, and chocolate coatings.

Soy protein products (e.g., meal), can be divided into soy flour concentrates and isolates which have both food/feed and industrial use. Soy flour and grits are often used in the manufacturing of meat extenders and analogs, pet foods, baking ingredients and other food products. Food products made from soy flour and isolate include baby food, candy products, cereals, food drinks, noodles, yeast, beer, ale, etc. Soybean meal in particular is commonly used as a source of protein in livestock feeding, primarily swine and poultry. Feed uses thus include, but are not limited to, aquaculture feeds, bee feeds, calf feed replacers, fish feed, livestock feeds, poultry feeds and pet feeds, etc.

Whole soybean products can also be used as food or feed. Common food usage includes products such as the seed, bean sprouts, baked soybean, full fat soy flour used in various products of baking, roasted soybean used as confectioneries, soy nut butter, soy coffee, and other soy derivatives of oriental foods. For feed usage, hulls are commonly removed from the soybean and used as feed.

Soybeans additionally have many industrial uses. One common industrial usage for soybeans is the preparation of binders that can be used to manufacture composites. For example, wood composites may be produced using modified soy protein, a mixture of hydrolyzed soy protein and PF resins, soy flour containing powder resins, and soy protein containing foamed glues. Soy-based binders have been used to manufacture common wood products such as plywood for over 70 years. Although the introduction of urea-formaldehyde and phenol-formaldehyde resins has decreased the usage of soy-based adhesives in wood products, environmental concerns and consumer preferences for adhesives made from a renewable feedstock have caused a resurgence of interest in developing new soy-based products for the wood composite industry.

Preparation of adhesives represents another common industrial usage for soybeans. Examples of soy adhesives include soy hydrolyzate adhesives and soy flour adhesives. Soy hydrolyzate is a colorless, aqueous solution made by reacting soy protein isolate in a 5 percent sodium hydroxide solution under heat (120° C.) and pressure (30 psig). The resulting degraded soy protein solution is basic (pH 11) and flowable (approximately 500 cps) at room temperature. Soy flour is a finely ground, defatted meal made from soybeans. Various adhesive formulations can be made from soy flour, with the first step commonly requiring dissolving the flour in a sodium hydroxide solution. The strength and other properties of the resulting formulation will vary depending on the additives in the formulation. Soy flour adhesives may also potentially be combined with other commercially available resins.

Soybean oil may find applications in a number of industrial uses. Soybean oil is the most readily available and one of the lowest-cost vegetable oils in the world. Common industrial uses for soybean oil include use as components of anti-static agents, caulking compounds, disinfectants, fungicides, inks, paints, protective coatings, wallboard, anti-foam agents, alcohol, margarine, paint, ink, rubber, shortening, cosmetics, etc. Soybean oils have also for many years been a major ingredient in alkyd resins, which are dissolved in carrier solvents to make oil-based paints. The basic chemistry for converting vegetable oils into an alkyd resin under heat and pressure is well understood to those of skill in the art.

Soybean oil in its commercially available unrefined or refined, edible-grade state, is a fairly stable and slow-drying oil. Soybean oil can also be modified to enhance its reactivity under ambient conditions or, with the input of energy in various forms, to cause the oil to copolymerize or cure to a dry film. Some of these forms of modification have included epoxidation, alcoholysis or tranesterification, direct esterification, metathesis, isomerization, monomer modification, and various forms of polymerization, including heat bodying.

Solvents can also be prepared using soy-based ingredients. For example, methyl soyate, a soybean-oil based methyl ester, is gaining market acceptance as an excellent solvent replacement alternative in applications such as parts cleaning and degreasing, paint and ink removal, and oil spill remediation. It is also being marketed in numerous formulated consumer products including hand cleaners, car waxes and graffiti removers. Methyl soyate is produced by the transesterification of soybean oil with methanol. It is commercially available from numerous manufacturers and suppliers. As a solvent, methyl soyate has important environmental- and safety-related properties that make it attractive for industrial applications. It is lower in toxicity than most other solvents, is readily biodegradable, and has a very high flash point and a low level of volatile organic compounds (VOCs). The compatibility of methyl soyate is excellent with metals, plastics, most elastomers and other organic solvents. Current uses of methyl soyate include cleaners, paint strippers, oil spill cleanup and bioremediation, pesticide adjuvants, corrosion preventives and biodiesel fuels additives.

### VI. Kits

Any of the compositions described herein may be comprised in a kit. In a non-limiting example, a composition for the detection of a polymorphism as described herein and/or additional agents, may be comprised in a kit. The kits may thus comprise, in suitable container means, a probe or primer for detection of the polymorphism and/or an additional agent of the present invention. In specific embodiments, the kit will allow detection of at least one allele associated increased yield, for example, by detection of polymorphisms in such alleles and/or otherwise in linkage disequilibrium with the allele(s).

The kits may comprise a suitably aliquoted agent composition(s) of the present invention, whether labeled or unlabeled for any assay format desired to detect such alleles. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. The kits of the present invention also will typically include a means for containing the detection composition and any other reagent containers in close confinement for commercial sale. Such containers may include injection or blow-molded plastic containers in which the desired vials are retained.

When the components of the kit are provided in one and/or more liquid solutions, the liquid solution may be an aqueous one, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means. The container means will generally include at least one vial, test tube, flask, bottle, syringe and/or other container means, into which the composition for detecting a null allele is placed, preferably, suitably aliquoted. The kits may also comprise a second container means for containing a sterile buffer and/or other diluent.

### VII. Definitions

In the description and tables which follow, a number of terms are used. In order to provide a clear and consistent understanding of the specification and claims, the following definitions are provided:

A: When used in conjunction with the word “comprising” or other open language in the claims, the words “a” and “an” denote “one or more.”

Agronomically Elite: As used herein, means a genotype that has a culmination of many distinguishable traits such as seed yield, emergence, vigor, vegetative vigor, disease resistance, seed set, standability and threshability which allows a producer to harvest a product of commercial significance.

Allele: Any of one or more alternative forms of a gene locus, all of which one of the forms of the gene locus relate to a trait or characteristic. In a diploid cell or organism, the two alleles of a given gene occupy corresponding loci on a pair of homologous chromosomes.

Backcrossing: A process in which a breeder repeatedly crosses hybrid progeny, for example a first generation hybrid (F₁), back to one of the parents of the hybrid progeny. Backcrossing can be used to introduce one or more single locus conversions from one genetic background into another.

Commercially Significant Yield: A yield of grain having commercial significance to the grower represented by an actual grain yield of at least 95% of the check lines MV0038 and DKB23-51 when grown under the same conditions.

Crossing: The mating of two parent plants.

Cross-pollination: Fertilization by the union of two gametes from different plants.

F₁ Hybrid: The first generation progeny of the cross of two non-isogenic plants.

Genotype: The genetic constitution of a cell or organism.

High yield: A yield of grain having commercial significance to the grower represented by an actual grain yield of at least 103% of the check lines MV0038 and DKB23-51 when grown under the same conditions.

INDEL: Genetic mutations resulting from insertion or deletion of nucleotide sequence.

Industrial use: A non-food and non-feed use for a soybean plant. The term “soybean plant” includes plant parts and derivatives of a soybean plant.

Linkage: A phenomenon wherein alleles on the same chromosome tend to segregate together more often than expected by chance if their transmission was independent.

Marker: A readily detectable phenotype, preferably inherited in codominant fashion (both alleles at a locus in a diploid heterozygote are readily detectable), with no environmental variance component, i.e., heritability equal to 1.

Non-transgenic mutation: A mutation that is naturally occurring, or induced by conventional methods (e.g. exposure of plants to radiation or mutagenic compounds), not including mutations made using recombinant DNA techniques.

Phenotype: The detectable characteristics of a cell or organism, which are the manifestation of gene expression.

Quantitative Trait Loci (QTL): Quantitative trait loci (QTL) refer to genetic loci that control to, some degree, numerically representable traits that are usually continuously distributed.

SNP: Refers to single nucleotide polymorphisms, or single nucleotide mutations when comparing two homologous sequences.

Stringent Conditions: Refers to nucleic acid hybridization conditions of 5×SSC, 50% formamide and 42° C.

Substantially Equivalent: A characteristic that, when compared, does not show a statistically significant difference (e.g., p=0.05) from the mean.

Tissue Culture: A composition comprising isolated cells of the same or a different type or a collection of such cells organized into parts of a plant.

Transgene: A genetic locus comprising a sequence which has been introduced into the genome of a soybean plant by transformation.

### VIII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Phenotypic Yield Marker

Five breeding populations were evaluated for yield and pubescence color. Table 1 summarizes the breeding populations and phenotype. The average yield of soybean with light tawny pubescence was 0.6 to 1.7 bu/a greater than yields of soybeans with other pubescence colors (Tables 2-3).

TABLE 1
Breeding populations

| | | Phenotype of |
|---|---|---|
| Population | Parents | Parents* |
| 1 | MV0080/MV0081 | GxLt |
| 2 | MV0082/MV0029 | LtxG |
| 3 | MV0082/MV0083 | LtxG |
| 4 | MV0080/MV0084 | GxLt |
| 5 | MV0085/MV0081 | TxLt |

*G = gray, Lt = light tawny, T = tawny

TABLE 2
Agronomic characteristics associated with pubescence
across breeding populations 1-4

| | No. of | Yield | Maturity | Plant Height |
|---|---|---|---|---|
| Phenotype | Individuals | (Bu/A) | (d) | (in) |
| Light Tawny | 169 | 63.51 | 21.56 | 39.20 |
| Mixed Pubescence | 176 | 62.89 | 21.57 | 39.55 |
| Gray | 256 | 62.74 | 21.27 | 38.89 |
| Tawny | 150 | 61.8 | 22.12 | 40.09 |

TABLE 3
Agronomic characteristics associated with pubescence
across breeding populations 1-5

| | No. of | Yield | Maturity | Plant Height |
|---|---|---|---|---|
| Phenotype | Individuals | (Bu/A) | (d) | (in) |
| Light Tawny | 215 | 62.83 | 21.37 | 39.83 |
| Mixed Pubescence | 218 | 62.19 | 21.48 | 39.23 |
| Tawny | 250 | 61.77 | 22.22 | 39.75 |

Plant maturity and plant height have an effect on grain yield. A delay in maturity or an increase in plant height generally increases yield. Therefore, it is critical to evaluate yield in conjunction with plant maturity and plant height to assure the increase in yield is not attributed to plant maturity or plant height. Data in Tables 2-3 shows that pubescence color does not appear to be associated with plant maturity or plant height.

### Example 2

### Identifying Genomic Regions Associated with Pubescence and Yield

One thousand, four hundred single nucleotide polymorphism (SNP) markers, randomly distributed across the 20 linkage groups of the soybean genetic linkage map, were used to identify SNP markers tightly linked with pubescence. Three hundred and sixty-three soybean varieties were phenotyped and fingerprinted. Two loci, Td locus and T locus, were identified to be associated with pubescence color. Td locus is located on linkage group N from 107-112 cM (Table 4). T locus is located on linkage group C2 from 88-91 cM (Table 5). A list of associated molecular markers that may be used for marker assisted selection are listed in Table 6. Yield was found to be associated with similar regions as Td and T loci (Tables 7 and 8).

TABLE 4
Examples of molecular markers associated with Td locus and distribution
of pubescence phenotype in 361 soybean varieties

| | SEQ ID NO: | | | | | | |
|---|---|---|---|---|---|---|---|
| | 1 | | 17 | | 7 | | |
| | Position (cM) | | | | | | Total |
| | 107.4 | | 112 | | 112 | | Number |
| Alleles | TT | CC | AA | GG | GG | CC | of Varieties |
| Gray | 88 | 36 | 84 | 31 | 29 | 90 | 139 |
| Light Tawny | 54 | 0 | 1 | 52 | 52 | 2 | 57 |
| Tawny | 90 | 65 | 97 | 49 | 54 | 101 | 165 |

TABLE 5
Examples of molecular markers associated with T locus and distribution
of pubescence phenotype in 361 soybean varieties

| | SEQ ID NO: | | | | | | | | |
|---|---|---|---|---|---|---|---|---|---|
| | 20 | | 24 | | 19 | | 23 | | |
| | Position (cM) | | | | | | | | Total |
| | 89 | | 89 | | 89 | | 89 | | Number |
| Alleles | AA | TT | TT | CC | AA | GG | CC | AA | of Varieties |
| Gray | 3 | 129 | 8 | 113 | 11 | 121 | 26 | 93 | 139 |
| Light Tawny | 55 | 0 | 55 | 0 | 55 | 1 | 54 | 0 | 57 |
| Tawny | 140 | 20 | 128 | 2 | 128 | 31 | 122 | 11 | 165 |

TABLE 6
Molecular Markers for selection of pubescence

| Locus | LG | Position | SEQ | Primer | Primer | Probe | Probe |
|---|---|---|---|---|---|---|---|
| Td | N | 107.4 | 1 | 27 | 28 | 79 | 80 |
| Td | N | 111.6 | 2 | 29 | 30 | 81 | 82 |
| Td | N | 111.6 | 3 | 31 | 32 | 83 | 84 |
| Td | N | 111.6 | 4 | 33 | 34 | 85 | 86 |
| Td | N | 111.6 | 5 | 35 | 36 | 87 | 88 |
| Td | N | 111.6 | 6 | 37 | 38 | 89 | 90 |
| Td | N | 111.6 | 7 | 39 | 40 | 91 | 92 |
| Td | N | 111.6 | 8 | 41 | 42 | 93 | 94 |
| Td | N | 110.8 | 9 | 43 | 44 | 95 | 96 |
| Td | N | 111.6 | 10 | 45 | 46 | 97 | 98 |
| Td | N | 111.6 | 11 | 47 | 48 | 99 | 100 |
| Td | N | 111.6 | 12 | 49 | 50 | 101 | 102 |
| Td | N | 111.6 | 13 | 51 | 52 | 103 | 104 |
| Td | N | 111.6 | 14 | 53 | 54 | 105 | 106 |
| Td | N | 111.6 | 15 | 55 | 56 | 107 | 108 |
| Td | N | 111.6 | 16 | 57 | 58 | 109 | 110 |
| Td | N | 111.6 | 17 | 59 | 60 | 111 | 112 |
| T | C2 | 88.3 | 18 | 61 | 62 | 113 | 114 |
| T | C2 | 89.0 | 19 | 63 | 64 | 115 | 116 |
| T | C2 | 89.0 | 20 | 65 | 66 | 117 | 118 |
| T | C2 | 89.0 | 21 | 67 | 68 | 119 | 120 |
| T | C2 | 89.0 | 22 | 69 | 70 | 121 | 122 |
| T | C2 | 89.0 | 23 | 71 | 72 | 123 | 124 |
| T | C2 | 89.0 | 24 | 73 | 74 | 125 | 126 |
| T | C2 | 89.7 | 25 | 75 | 76 | 127 | 128 |
| T | C2 | 89.7 | 26 | 77 | 78 | 129 | 130 |

TABLE 7
Yield associated with Td region (LG N 107-112cM)

| | Cross | | | |
|---|---|---|---|---|
| | | MV0088/ | MV0090/ | MV0092/ |
| | MV0086/MV0087 | MV0089 | MV0091 | MV0091 |
| SEQ ID NO: | 17 | 17 | 9 | 17 |
| LG | N | N | N | N |
| Pos | 111.6 | 111.6 | 110.8 | 111.6 |
| Allele | G | G | A | G |
| Trait | YLD | YLD | YLD | YLD |
| P-value | 2.61629E−05 | 0.000249805 | 0.014459157 | 0.038693115 |
| F-Statistic | 18.99232815 | 14.26895227 | 6.112113606 | 4.359644085 |
| Marker Effect | 0.97656912 | −0.729604654 | −0.190825752 | 0.175866326 |
| Fav Parent | MV0087 | MV0088 | MV0090 | MV0092 |

TABLE 8
Yield associated with T region (LG C2 88-91 cM)

| | Cross | | | |
|---|---|---|---|---|
| | | MV0027/ | MV0027/ | MV0095/ |
| | MV0093/MV0094 | MV0038 | MV0038 | MV0096 |
| SEQ ID | 19 | 19 | 24 | 20 |
| NO: | | | | |
| LG | 9 | 9 | 9 | 9 |
| Pos | 89 | 89 | 89 | 89 |
| Allele | G | G | C | T |
| Trait | YLD | YLD | YLD | YLD |
| P-value | 0.008805644 | 0.004170851 | 0.009925501 | 0.008644723 |
| F-Statistic | 7.047614476 | 8.435907129 | 6.802063372 | 7.077241568 |
| Marker Effect | −0.549706 | −0.394396 | −0.373612 | −0.323932 |
| Fav Parent | MV0094 | MV0038 | MV0038 | MV0096 |

### Example 3

### Association of Pubescence Color and Branching of Stems

Pubescence color and lateral branching was evaluated for 66 soybean plants. Plants were rated 1-3 for branching (1=modest branching, 2=moderate branching, 3=profuse branching). Light tawny soybeans had significantly higher branching than either gray or tawny soybeans (Table 9-10). An increase in branching may be associated with higher yield. High density cultivation also requires optimization of lateral branching. Another target for yield improvement has therefore been the adaptation of plant architecture to current agricultural practices (Van Camp, 2005). The association of branching with pubescence color will assist in phenotyping the plant at an earlier stage.

TABLE 9
One way ANOVA for effect of pubescence on lateral branching

| Source | DF | Sum of Squares | Mean Square | F-value | Pr > F |
|---|---|---|---|---|---|
| Model | 2 | 4.98 | 2.49 | 3.60 | 0.0332 |
| Error | 63 | 43.64 | 0.69 | | |
| Corrected Total | 65 | 48.62 | | | |
| | | Type III: | | | |
| Source | DF | Sum of Squares | Mean Square | F-value | Pr > F |
| Pubescence | 2 | 4.98 | 2.49 | 3.6 | 0.0332 |

Table 10a:Least Squared means of branch for each
pubescence phenotype

| Pubescence | LS Means: Branching |
|---|---|
| Gray | 2.02 |
| Light Tawny | 2.50 |
| Tawny | 1.67 |

Table 10b: Pairwise comparisons of LS Means for effect of
pubescence on lateral branching

| | Gray | Light Tawny (P- | Tawny |
|---|---|---|---|
| Pubescence | (P-value) | value) | (P-value) |
| Gray | — | 0.0607 | 0.1966 |
| (P-value) | | | |
| Light Tawny | 0.0607 | — | 0.0109 |
| (P-value) | | | |
| Tawny | 0.1966 | 0.0109 | — |
| (P-value) | | | |

### Example 4

### Selecting for Light Tawny Phenotype

Individual markers were highly correlated with the loci T and Td. Alleles for NS0098757 and NS0113988 associated with locus Td are highly conserved in light tawny varieties, but the alleles are also found in gray varieties. The two markers are approximately 4cM apart. The allelic combination of TTGG or CTGG account for 89% of light tawny varieties, 17% of gray varieties and only 4% of tawny varieties in a screen of 363 soybean varieties (Table 11). The allele for SEQ ID NO: 21 associated with locus Tis highly conserved in light tawny varieties. Moreover, when the 363 soybean varieties were screened with SEQ ID NO: 7 and 12 for locus Td and SEQ ID NO: 21 associated with locus T, only 2% of the gray varieties and 3% of the tawny varieties had the same genotype as the light tawny varieties. Therefore, screening for both loci T and Td is more predictive for pubescence phenotype and increases in grain yield. Furthermore, several varieties have increased grain yield and the light tawny genotype, but are not light tawny. Therefore the selection of varieties with haplotype for locus Td with the selection for the dominant allele of locus T is predictive of increases in grain yield independent of pubescence color.

TABLE 11
Screening for light tawny phenotype

| Alleles | | | | | |
|---|---|---|---|---|---|
| Locus Td | Locus Td | Locus T | Phenotype | | |
| SEQ ID | SEQ ID | SEQ ID | | Light | |
| NO: 7 | NO: 12 | NO: 21 | Tawny | Tawny | Gray |
| GG | T_{—} | AA | 5 | 51 | 3 |
| G_{—} | TT | TT | 0 | 0 | 30 |
| GG | CC | _{——} | 44 | 0 | 6 |
| CC | _{——} | _{——} | 101 | 2 | 90 |
| ** | TT | ** | 3 | 3 | 3 |
| ** | ** | ** | 2 | 0 | 2 |
| GG | ** | AA | 3 | 1 | 0 |
| ** | CC | ** | 2 | 0 | 1 |
| ** | ** | TT | 1 | 0 | 3 |
| ** | ** | AA | 3 | 0 | 0 |

### Example 5

### Breeding Strategies for Increased Yield

Marker assisted selection is used for gene enrichment or fixation in populations segregating at the T and/or Td loci. There are several mapped SNPs in the regions of both the T and Td loci. When parents of a cross are polymorphic for either T or Td, they are useful for screening progeny for the pubescence color traits. A group of markers at each loci display linkage disequilibrium (LD) with the pubescence color alleles (Table 12). Seed is screened with polymorphic SNP markers. The genotypic and phenotypic data are compared to identify a loci associated with pubescence color or yield. The statistical significance of pubescence color markers association for T and Td loci is assessed using QTLCartographer (Basten et al. 1995). This analysis fits the data to the simple linear regression model:

y=b0+b1 x+e

The results give the estimates for b0, b1 and the F statistic for each marker. Whether or not a marker is linked to a QTL is determined by evaluating whether b1 is significantly different from zero. The F statistic compares the hypothesis H0: b1=0 to an alternative H1: b1≠0. The pr(F) is a measure of how much support there is for H0. A smaller pr(F) indicates less support for H0 and thus more support for H1. Significance at the 5%, 1%, 0.1% and 0.01% levels are indicated by *, **, *** and ****, respectively. When two soybean lines differ for one of the pubescence alleles, the markers with the greater LD are the most likely to be polymorphic. These marker alleles are predictive of pubescence phenotype.

TABLE 12
Markers significantly associated with Light tawny phenotype

| | | | Light Tawny | |
|---|---|---|---|---|
| SEQ ID NO: | LG | Position | allele | LD |
| 1 | N | 107.4 | TT | ** |
| 18 | N | 111.6 | GG | ** |
| 7 | N | 111.6 | GG | ** |
| 20 | C2 | 89.0 | GG | ** |
| 21 | C2 | 89.0 | TT | *** |
| 24 | C2 | 89.0 | AA | * |
| 25 | C2 | 89.0 | CC | ** |

### Cross Strategy:

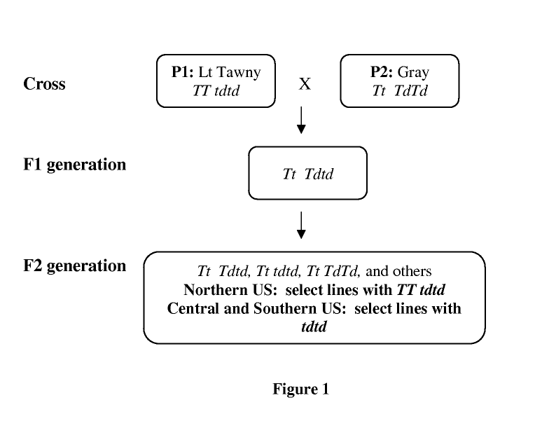

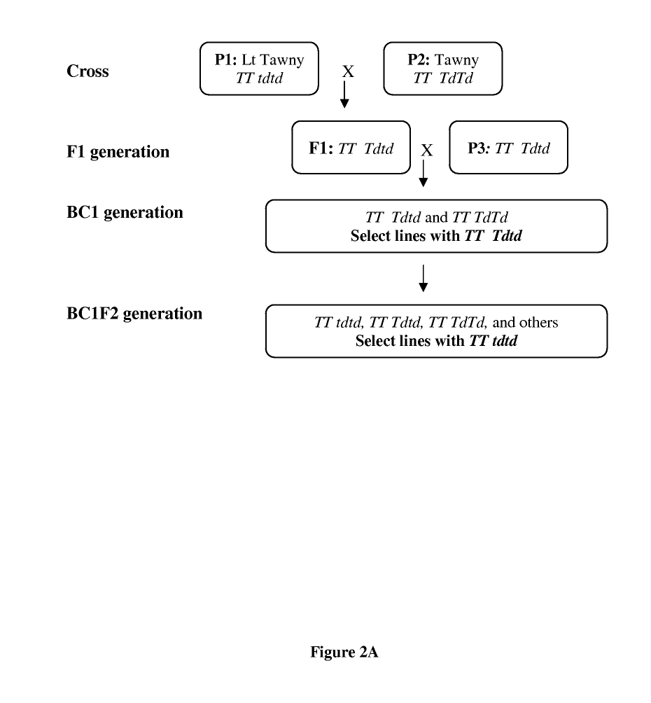

This strategy is useful for crossing any phenotype, for example crossing a light tawny line with a gray line (FIG. 1). F₂ plants are screened with markers associated with td allele on LG N. F₂ plants identified with the tdtd genotype are selected. If desired, the plant also could be selected for T allele on C2.

Backcross Strategy: Tawny (TT TdTd)×Light Tawny (TT tdtd)

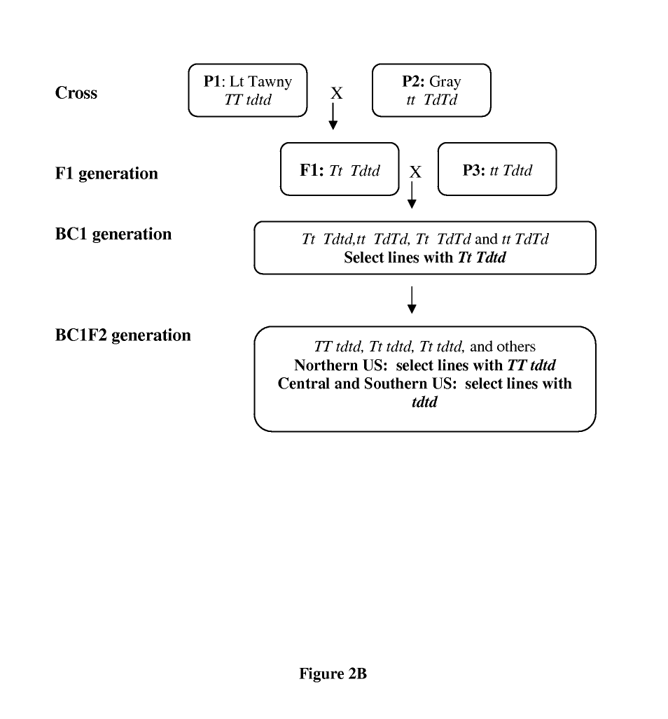

A light tawny line is crossed and backcrossed to a tawny line (FIG. 2A). The BC₁ plants are screened with the markers on LG N. BC₁ plants (˜50%) are selected with markers associated with Td markers. The BC₁F₂ seed is screened with markers associated with Td markers. Individual seeds with the tdtd genotype are selected for advancement.

Backcross Strategy: Gray (U TdTd)×Light Tawny (TT tdtd)

A light tawny line is crossed and backcrossed to a gray line (FIG. 2B). The BC₁ plants are screened with the markers on LG N and LG C2. The BC₁ plants (˜25%) are selected with markers associated with the Tt Tdtd genotype. The BC₁F₂ seed are screened and selected for the light tawny (tdtd) genotype. If desired, the plant also could be selected for T allele on C2.

### Example 6

### Purification of Breeding Lines for Commercialization

Soybean breeding lines must be phenotypically uniform prior to commericialzation. Varieties are selected to be phenotypically homogenous and uniform for such traits as flower color, branching, hilum color and pubescence. Zabella and Vodkin (2007) cloned and sequenced the W1 locus. The mutation is a rearrangement leading to a small (65 bp) insertion of tandem repeats in exon 3 that truncates the translation product prematurely. Soybeans have an all white flower phenotype in the presence of the insertion. The T locus has also been cloned and the causel sequence polymorphisms identified (Toda et al. 2002; Zabella and Vodkin 2003). The development of allelic specific markers for the purple/white flower color (w1 locus) grey/tawny pubescence locus (T locus) and the tawny/light tawny pubescence locus (Td locus) are valuable for the purification of soybean varieties. Furthermore, the branching type can be predicted by the association with pubescence color. For example, segregating soybean lines could be assayed for W1, T, and Td loci relatively cheaply and quickly through the use of linked molecular markers or preferably, allelic specific markers as seed instead of pheontypically at mature plant stage. Subsequently, seeds with similar genotypes/ phenotypes could be separated into bulks that are pure enough for commercial product. Implementation of this strategy reduces the commercialization time by a year or more for many lines. Additionally, the process helps characterize soybean lines, as the lines could be characterized at any stage of the life cycle (including seed).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,992,375
U.S. Pat. No. 5,015,580
U.S. Pat. No. 5,024,944
U.S. Pat. No. 5,416,011
U.S. Pat. No. 5,545,545
U.S. Pat. No. 5,637,785
U.S. Pat. No. 6,031,154
U.S. Pat. No. 6,140,085
U.S. Pat. No. 6,184,440
U.S. Pat. No. 6,486,383
U.S. Pat. No. 6,774,284
Allard, In: Principles of Plant Breeding, John Wiley & Sons, NY, 50-98, 1960.
Anonymous, Seedquest, 17 Feb. 2007
Beachy et al., Ann. rev. Phytopathol. 28:451, 1990.
Boerma and Moradshahi, Crop Sci., 15:858-861, 1975.
Borthwick and Parker, Bot. Gaz., 100:374-387, 1938.
Brim and Stuber, Crop Sci., 13:528-530, 1973.
Charest et al., Plant Cell Rep. 8:643 1990.
Christianson et al., Science, 222:632-634, 1983.
Comai et al., Nature 317:741-744, 1985.
Concibido et al., Theor. Appl. Genet. 106:575-582, 2003.
Criswell and Hume, Crop Sci., 12:657-660, 1972.
Eichholtz et al., Somatic Cell Mol. Genet. 13:67, 1987.
Elliot et al., Plant Molec. Biol. 21:515 (1993
European Appln. 0 242 246
European Appln. 0640141
European Appln. 0797673
Fehr, In: Soybeans: Improvement, Production and Uses, 2nd Edition, Manograph., 16:249, 1987a.
Fehr, In: Theory and Technique, and Crop Species Soybean, Iowa State Univ., Macmillian Pub. Co., NY, (1)(2):360-376, 1987b.
Fehr, In: Hybridization of Crop Plants, Fehr and Hadley (Eds.), Am. Soc. Agron. and Crop Sci. Soc. Am., Madison, Wis., 90-599, 1980.
Finer et al., In: Soybean: Genetics, Molecular Biology and Biotechnology, CAB Intl., Verma and Shoemaker (ed), Wallingford, Oxon, UK, 250-251, 1996.
Fisher et al., Plant Physiol., 102(3):1045-1046, 1993.
Fraley et al., Proc. Natl. Acad. Sci. USA, 80:4803, 1983.
Geiser et al., Gene, 48:109, 1986.
Gordon-Kamm et al., Plant Cell, 2:603-618, 1990.
Guzman et al., Crop Sci 47:111-122, 2007.
Hamner, In: The Induction of Flowering: Some Case Histories, Evans (ed), Cornell Univ. Press, Ithaca, N.Y., 62-89, 1969.
Hartweck et al., In Vitro Cell. Develop. Bio., 24:821-828, 1988.
Iwashina et al., J Heredity, 97:438-443, 2006.
Jones et al., Science, 266:789, 1994.
Kisha et al. Crop Sci. 37:1317-1325, 1997.
Knutzon et al., Proc. Natl. Acad. Sci. USA, 89:2624, 1992.
Lee et al., EMBO J., 7:1241, 1988.
Logemann et al., Bio/Technology, 10:305, 1992.
Marshall et al., Theor. Appl. Genet., 83:435, 1992.
Martin et al., Science, 262:1432, 1993.
Miki et al., Theor. Appl. Genet., 80:449, 1990.
Mindrinos et al., Cell, 78:1089, 1994.
Morrison et al. Agron J 89: 218-221, 1997.
Orf et al. Crop Sci. 39:1642-1651, 1999.
Orf et al., In: Soybeans: Improvement, production and uses. 3rd ed. Agron. Monogr. 16. ASA, CSSA, and SSSA, Madison, Wis. p. 417-450, 2004.
PCT Appln. US93/06487
PCT Appln. WO93/19181
PCT Appln. WO96/30517
Poehlman and Sleper, In: Breeding Field Crops, Iowa State University Press, Ames, 1995.
Przibila et al., Plant Cell, 3:169, 1991.
Raper and Kramer, In:. J. R. Wilcox (ed.) Soybeans: Improvement, production, and uses. 2nd. ed. Agron. Monogr. 16. ASA, CSSA, and SSSA, Madison, Wis. p. 589-641, 1987.
Shah et al., Science, 233:478, 1986.
Shanmugasundaram and Tsou, Crop Sci., 18:598-601, 1978.
Shibles et al., In: Crop Physiology, Some Case Histories, Evans (ed), Cambridge Univ. Press, Cambridge, England, 51-189, 1975.
Shirley, Trends Plant Sci 1:377-382, 1996.
Shirley, Plant Physiol 126:485-493, 2001.
Shiroza et al., J. Bacteol., 170:810, 1988.
Sinclair and Backman, In: Compendium of Soybean Diseases, 3^{rd} Ed. APS Press, St. Paul, Minn., p. 106, 1989.
Simmonds, In: Principles of crop improvement, Longman, Inc., NY, 369-399, 1979.
Sneep and Hendriksen, In: Plant breeding perspectives, Wageningen (ed), Center for Agricultural Publishing and Documentation, 1979.
Søgaard et al., J. Biol. Chem., 268(30):22480-22484, 1993.
Stalker et al., Science, 242:419-423, 1988.
Steinmetz et al., Mol. Gen. Genet., 20:220, 1985.
Sunada and Ito, S22:34, 1982.
Thompson et al., Crop Sci. 38:1348-1355, 1998.
Thorne and Fehr, Crop Sci. 10:652-655, 1970.
Toda et al. Plant Mol. Biol. 50: 187-196., 2002.
Toda et al. Crop Sci 45:2212-2217, 2005.
Van Camp. Cur. Opin. Biotechnol.16: 147-153, 2005.
Vanden Elzen et al., Plant Mol. Biol., 5:299, 1985.
Wright et al., Plant Cell Reports, 5:150-154, 1986.
Zabella and Vodkin, Genetics 163: 295-309, 2003.
Zabala and Vodkin, Crop Sci. 47: S113-S124, 2007.
Zang and Smith, Pl Physiol 108: 961-968, 1995.

What is claimed is:

1. A method of introgressing an allele into a soybean plant comprising
(A) crossing at least one first soybean plant comprising a nucleic acid molecule selected from the group consisting of SEQ ID NO:1 through SEQ ID NO: 26 with at least one second soybean plant in order to form a segregating population,
(B) genotyping at least one soybean plant in the segregating population with respect to a soybean genomic nucleic acid marker selected from the group SEQ ID NO:1 through SEQ ID NO: 26, and
(C) selecting from the segregation population at least one soybean plant comprising at least one nucleic acid molecule selected from the group consisting of SEQ ID NO: 1 through SEQ ID NO: 26.

2. The method according to claim 1, wherein said selected one or more soybean plants further comprises a second sequence selected from the group consisting of SEQ ID NO: 1 to SEQ ID NO: 26.

3. The method according to claim 2, wherein said selected one or more soybean plants further comprises a third sequence selected from the group consisting of SEQ ID NO: 1 to SEQ ID NO: 26.

4. The method according to claim 1, wherein said selected one or more soybean plants exhibit increased grain yield.

5. The method according to claim 1, wherein said selected one or more soybean plants exhibit an increased grain yield of at least 0.5 Bu/A.

6. The method according to claim 1, wherein said selected one or more soybean plants exhibit an increased grain yield of at least 1.0 Bu/A.

7. The method according to claim 1, wherein said selected one or more soybean plants exhibit an increased grain yield of at least 1.5 Bu/A.

8. The method according to claim 1, wherein said selected one or more soybean plants exhibit altered flavonoid synthesis.

9. The method according to claim 8, wherein said selected one or more soybean plants exhibit altered flower pigmentation, plant-microbe interactions, protection from UV radiation, symbiotic relationships between bacteria or fungi and plant root, disease resistance, insect resistance, and nodulation.

10. The method according to claim 8, wherein said selected one or more soybean plants exhibit increased human heath benefits with human consumption.

11. The method of claim 1, wherein genotyping is affected in step (B) by determining the allelic state of at least one of said soybean genomic DNA markers.

12. The method of claim 2, wherein said allelic state is determined by an assay which is selected from the group consisting of single base extension (SBE), allele-specific primer extension sequencing (ASPE), DNA sequencing, RNA sequencing, microarray-based analyses, universal PCR, allele specific extension, hybridization, mass spectrometry, ligation, extension-ligation, and Flap Endonuclease-mediated assays.

13. The method of claim 1, further comprising the step of crossing the soybean plant selected in step (C) to another soybean plant.

14. The method of claim 1, further comprising the step of obtaining seed from the soybean plant selected in step (C).

15. The method of claim 1, wherein at least one soybean plant in the segregating population is genotyped with respect to a soybean genomic DNA marker selected from the group consisting of SEQ ID NO:1 through SEQ ID NO: 26.

16. A method of introgressing an allele into a soybean plant comprising:
(A) crossing at least one plant with pubescence allele with at least one plant in order to form a segregating population;
(B) screening the segregating population with at least one nucleic acid marker to determine if one or more soybean plants from the segregating population contains the pubescence allele, wherein said pubescence allele is an allele selected from the group consisting of T or Td loci.

17. A method according to claim 16, where at least one of the markers is located within 30 cM of the pubescence allele.

18. A method according to claim 16, where at least one of the markers is located within 25 cM of the pubescence allele.

19. A method according to claim 16, where at least one of the markers is located within 20 cM of the pubescence allele.

20. A method according to claim 16, where at least one of the markers is located within 15 cM of the pubescence allele.

21. A method according to claim 16, where at least one of the markers is located within 10 cM of the pubescence allele.

22. A method according to claim 16, where at least one of the markers is located within 5 cM of the pubescence allele.

23. A method according to claim 16, where at least one of the markers is located within 2 cM of the pubescence allele.

24. A method according to claim 16, where at least one of the markers is located within 1 cM of the pubescence allele.

25. A soybean plant obtained from the method of claim 16, comprising a nucleic acid molecule selected from the group consisting of SEQ ID NO:1 through SEQ ID NO: 26.

26. The soybean plant according to claim 25, wherein the soybean plant exhibits a transgenic trait.

27. The soybean plant according to claim 26, wherein the transgenic trait is selected from the group consisting of herbicide tolerance, increased yield, insect control, fungal disease resistance, virus resistance, nematode resistance, bacterial disease resistance, mycoplasma disease resistance, modified oils production, high oil production, high protein production, germination and/or seedling growth control, enhanced animal and human nutrition, low raffinose, environmental stress resistance, increased digestibility, improved processing traits, improved flavor, nitrogen fixation, hybrid seed production, and/or reduced allergenicity.

28. The soybean plant according to claim 27, wherein the herbicide tolerance is selected from the group consisting of glyphosate, dicamba, glufosinate, sulfonylurea, bromoxynil and norflurazon herbicides.

29. The soybean plant according to claim 25, wherein the nucleic acid molecule is present as a single copy in the soybean plant.

30. The soybean plant according to claim 25, wherein the nucleic acid molecule is present in two copies in the soybean plant.

31. A substantially purified nucleic acid molecule selected from the group consisting of SEQ ID NO:1 through SEQ ID NO: 130 and complements thereof.

32. A soybean plant comprising pubescence locus Td.

33. A soybean plant comprising pubescence locus T and Td.

34. An isolated nucleic acid molecule for detecting a molecular marker representing a polymorphism in soybean DNA, wherein said nucleic acid molecule comprises at least 15 nucleotides that include or are immediately adjacent to said polymorphism, wherein said nucleic acid molecule is at least 90 percent identical to a sequence of the same number of consecutive nucleotides in either strand of DNA that include or are immediately adjacent to said polymorphism, and wherein said molecular marker is selected from the group consisting of SEQ ID NO:1 through SEQ ID NO: 26.

35. The isolated nucleic acid of claim 35, wherein said nucleic acid further comprises a detectable label or provides for incorporation of a detectable label.

36. The isolated nucleic acid of claim 36, wherein said detectable label is selected from the group consisting of an isotope, a fluorophore, an oxidant, a reductant, a nucleotide and a hapten.

37. The isolated nucleic acid of claim 37, wherein said detectable label is added to the nucleic acid by a chemical reaction or incorporated by an enzymatic reaction.

38. The isolated nucleic acid of claim 35, wherein said nucleic acid molecule comprises at least 16 or 17 nucleotides that include or are immediately adjacent to said polymorphism.

39. The isolated nucleic acid of claim 39, wherein said nucleic acid molecule comprises at least 18 nucleotides that include or are immediately adjacent to said polymorphism.

40. The isolated nucleic acid of claim 39 wherein said nucleic acid molecule comprises at least 20 nucleotides that include or are immediately adjacent to said polymorphism.

41. The isolated nucleic acid of claim 35, wherein said nucleic acid molecule hybridizes to at least one allele of said molecular marker under stringent hybridization conditions.

42. The isolated nucleic acid of claim 35, wherein said molecular markers are SEQ ID NO: 1 through SEQ ID NO: 17 and said nucleic acid is an oligonucleotide that is at least 90% identical to SEQ ID NO: 79 through SEQ ID NO: 112.

43. The isolated nucleic acid of claim 35, wherein said molecular markers are SEQ ID NO: 18 through SEQ ID NO: 26 and said nucleic acid is an oligonucleotide that is at least 90% identical to SEQ ID NO: 113 through SEQ ID NO: 130.

44. A set of oligonucleotides comprising:
(A) a pair of oligonucleotide primers wherein each of the primers comprises at least 12 contiguous nucleotides and wherein the pair of primers permit PCR amplification of a DNA segment comprising a molecular marker selected from the group consisting of SEQ ID NO:1 through SEQ ID NO: 26.
(B) at least one detector oligonucleotide that permits detection of a polymorphism in the amplified segment, wherein the sequence of the detector oligonucleotide is at least 95 percent identical to a sequence of the same number of consecutive nucleotides in either strand of a segment of maize DNA that include or are immediately adjacent to the polymorphism of step (A).

45. The set of oligonucleotides of claim 45, wherein said detector oligonucleotide comprises at least 12 nucleotides and either provides for incorporation of a detectable label or further comprises a detectable label.

46. The set of oligonucleotides of claim 46, wherein said detectable label is selected from the group consisting of an isotope, a fluorophore, an oxidant, a reductant, a nucleotide and a hapten.

47. The set of oligonucleotides of claim 45, wherein said detector oligonucleotide and said oligonucleotide primers hybridize to at least one allele of said molecular marker under stringent hybridization conditions.

48. The set of oligonucleotides of claim 45, further comprising a second detector oligonucleotide capable of detecting a second polymorphism of said molecular marker that is distinct from the polymorphism detected by a first detector oligonucleotide of said set of oligonucleotides.

49. The set of oligonucleotides of claim 45, further comprising a second detector oligonucleotide capable of detecting a distinct allele of the same polymorphism detected by a first detector oligonucleotide of said set of oligonucleotides.

50. A method of developing allele specific genetic markers for T, Td and W1 loci.

51. A method of purifying soybean lines comprising
(A) crossing at least one first soybean plant with at least one second soybean plant in order to form a segregating population,
(B) genotyping at least one soybean seed in the segregating population with respect to T, Td and W1 loci,
(C) selecting and bulking from the segregation population at least one soybean plant with similar genotypes with respect to T, Td and W1 loci.


SEQUENCE LISTING (130 sequences)

SEQ ID NO: 1 - DNA, 660 bp, Glycine max
agtatggaattttccatgacctaatgtccatcttgaaacgagttgagctcatattattatgcaattggttgcaaggaaaattaacatgatcaaatgcatttgtaactaaattgcaagcaacatgcttttttaacatactttctctgttggaggatacttctcatgttgatcttatttaataacctgggtccattctttattagtgggaccaatattaaataaattcatcaataaaaaaaggcagtatttctagtatcattttcgtgactttaaggtggccagtgaagcatccaccgacccccataatacctgaactgaatgcagtaagcaatgcactgtttttcaccagtgaagcattggcaatgttggtatttaattgtgcttgtgttattagtggtcaatttatttaacaaaagtgaggtagtgccagtagtttggttataaaatgcggcaaatgtattaggaaaacagggatcaattcattatgtgtgaagtagtgaagcacaaaacttgtttcaataaaattgttaacctggttgaaatcatcacaagagatctgtgtattggctataaaagctatgatacaattaaagggtaacataacataatgtagcaatggttaactaaactccttcagtgtgtttttaaatctttccaccc

SEQ ID NO: 2 - DNA, 811 bp, Glycine max
aagtctaaccggagtatttccatctttcatacactctgctagtgcaggaccgacaatagtgctgtgtgacataatggctgaaggatttgcctgtaatgttgcaggaatgtaggccatgtaaattttcaaccatataattctacaggtgatatcttggtcaaggcaagactgttcaagcacataccttggcaacagcttttatagcagataatgctcttcttctaacttcactggagtcatcatgtgtggatgatactagcaatgaaagaacatctttgtaaagaagtgtgtcagaaggatccacctgagatctataaagaaggagccttcccaatgccttggttgaagtttcgcgaagagggaactgttttatggaaaaattgaataaaatcggacaatagcaatataacaagaaacttgttagacttcagaaataaacacaatgaattctgaaaaaattatagaaataatctggattttagaccagatttgagatcaaaattaaacaacaaagggttttcccagagcaagagtttccaccttcaaaacacaagaaataataatctgacaccttaattgccatctacaagtctcacaacctatatatatcactataactatcacatcacaacatataattgccaaattcgaaattaatacacaaataactcctcaattacttccaaatacagaattaaggtgcttaattcagaaacatggcctaccaaaactaattgagtattaacataccttctcatccttcagggtgtctctaaggcaatcaacatagttgagaacaaggaagacgaac

SEQ ID NO: 3 - DNA, 792 bp, Glycine max
aatgcaaccctaaagaaactgtacccttgaggtacctcagaattctcgggttcacagattgcacagattcgaaaacaacatcactttcatgagacaaacttccaaaacagccttaagaaagagtaaacgagcctaagttttgagttaggttgatagaacctaaagcacgttttcgagaatgatcaatacacacctctcgcgcaagcaacagagtttcaacttcatcaacagagaatggatcaaatctactagtgatgagtgaaatcaatgattcatattaattagggagtccatcaagaatcaaattaagatgctcagaattggatactggttcactcttagaatgtgagtttgagtctaactcaatcccaaaagctagctcataggtgagggttgctccccacttatatactctattttggcattatatctaaccgatgtgggacttgggtttttttcaatacatcccttcacacccaacaattttggacttggtatatgaataatatgataggtgacccgttaatggatttaggataggctctgataccatcttagaatgtgagtttgagtctaactcaaccccaaaagctagttcataggtaagagttgttttccacttatatactctatcttggtactatctttagccgatgtggaaccttaggttttttttcaatattcaccaaaggaactgagcgcgttgactagattttgcacatgaagaagataataagagatggagcgattatcaagaagagatttcgaagctcattggcagcttgacaatct

SEQ ID NO: 4 - DNA, 830 bp, Glycine max
  unsure (1)..(830): unsure at all n locations, n = a, t, c, or g
tgattttaatttgtgttacattattaattaacgaatgatgaacttgcaagttgatgtgttttagtatgttgcaggatatattgtttgccatgcttgtggagataacggagctcattgtgacacaaaagatgttcttatatttggcggtgtggctcaagggggcgtgttgagagtcctacatcgggtggttaacgaacatgataagtactataatgatagcttatactggtctccttgaatttgctcatggtgcatcaactccactagaggattctacattcacccagcggttccggacaaatgaagtgaaagcaatatggagagaagaaaatttggcaaaattgaatgggcttgcagagaagagcacttgatttgtcaatcttgaggtgcactctcaaattaaacctttagtgtagaatatttagcgtgtatttgcaaatttgatgtgaagcaatttatgtttggatgtttgtgttctataagttaatagtaaaattcagtatgaaattttttatcaaacacaaaagctacctaaatttgtttcaatttataatcaattcttcaacattagactaaagatataaacatttatctacaattacattagctgtatttttacatgattatggatgacttaatgtagaatcaaacacggagtaaatttgtgcttagttttggcaaaaatttattttccatagtcaaacttaggcgggtaattttaatacattggntgtgttaaattcatatattaataaattaagatcaacgaccctgccttctttttctcagtttcagcatatttatgntcaaaaaggacttgactatatcat

SEQ ID NO: 5 - DNA, 816 bp, Glycine max
atgcctgcagccgcttgaaaccttgcagttgccacttgggaattttctgcatagacaaaaccattagagtatcagcatgacctaaaccagaatagtaaatactaaatataagtgtatactgtacctcaagtatttttacttactacagttaggttttgttagtttgttaactatagttagttgtcagttaggatagttgacaattctcactgtcaactatatatactcttcttataactgttttcaattaggttgaataacaatccgtttctcaatctctctcttctctctctctctctctcacacagtttctctgaattgtaaaatggtatacagagcttaggtcaaaaccaagcttttttcgtggcttccgcatcgactcctccttcctcctcaccgctgccatcgtcattggctagttcttccaaaatgttcacgcaatcaatttctcagaagttgaatgcaaagaactaccttctttggattcaacaggtgggatctgttattcatggtcatcatcttgaaggctacatcattaatcctcaaatttctcctaaatatgcttcggttgaagatcgcaattcagataaagtcaccagtgagtatcgtgtttggtacgaatagaatcagtttcttcttgtgtggttgtaatccattatttctggtgaaattcttcctcgcattgttggttgtcaatcgccgtggcagttatgggatcgcatccaatcacattttcaatctcactcacgcgaagatttgtcaagcttgcaatgagcttcgaaatctctcttgataatcgctccatctcttattatc

SEQ ID NO: 6 - DNA, 850 bp, Glycine max
tgcctgcagctgcagcttcagccataaaattctgcaccacatattcataattgtttcagttgattaccatgcaaactaaacacgcaacgcaagtgcatgtttggtttaacttattttgaagaaataaggactcagtttgtttaaacttatttattgaaataagtgtttattttaataaaataagcagttttctatttttttagtgtgtttaactaaactgtttctgcttaaaaaaaataattttatgtttattttaagaagtaaatcttatctgtttcttaaaaaatcgcttataaaaaaagcacttattttaaagttgttatttttaagtttaaacaaagtcacccaataacttattcttactaatagaaatttttgaaaaataagcgactgttatctaaatttaagtattacaaacattcattaaatcagttcaaacattgacaacataatccagaaaaattagcaacaagctcagagaaccttcagtgtctctatgggaacaaaataagaaaggatagaaaatgaaatgagtaagcaatgataattttctcacgtcatacaaatgaggggggtgaataggaagaattgaataaaaacattttacaattacaaatttactcccaacttttttgattgggacaatactttgggaaagacaactttgtaatttagaaattcaacaagctaatacccctacacttcctctccatttttagttaaaaaattagtcttgatgaaaacgaactctggtgcaaaatgaatattttatgtcccaatgaagactacaaacagattttactgaataatgtcatattaagattaaaaaaactgcagcatcttacggatg

SEQ ID NO: 7 - DNA, 839 bp, Glycine max
aaatataaaattaaagtagatctgtaattaataaattatagcttacatcacgtgtatcacctttctagctaggttgttaatctaagttcggaactgctgtttcagcacatttttttgctgtttggccaggctagagaagttgtctagttaatcaccatgaaattaagcatttgaaaagttatttctagcacaaacagtttacaaggtatatatatatagcataaataaattgaagaaaaatgaagccgacgaatcactatagcaagaagaaaagagaattgaatttgtttgattgctttataagcaagccgtagtatatatataatgctgaaaacacggggaaagtttattaattgcagcaaaagcatctttgataattaaagaaagacatgaatcaaagcattcaaatacattaatctctctcatgaacctaaaaaatcacatctaatgaaaggtccttgattagagaaaaagaaggtttgacaaatgaaacattagtgaagagtctacatcgttaaaaaaggaaaaatccacccacatgtccacactagctagccacacactcattattccaccacacggaacaaaagcagacaaaaagaaatcaaattggaattagtaataggaaataaaaaggcatttggagacaattaaattaatacagagggagtcctaaaggcaaaaacatcattaattaagatgaattgcaagtatagtcaaaactgacacatttcttttcaaactttctttttttctttgcatcattcataaagtaataagccagcagataaagccattccacaaacgaactaaaacattctttcttgcactaaagagca

SEQ ID NO: 8 - DNA, 966 bp, Glycine max
aaggataggaagtagaaaatctaccagaaccttcccaaatggcaaggcagccacatgcaggaaaattgtaattgagttctagaaatcattgaaatactgcatctccttatgaatgtgaaattgtgaggtatctctcctttattggaaataattctgctacaattaattagtaacaacaaaaaaaatgtgggttaagcagtagctgtacaatcccttctagaagacaagctgatggacttgggtacatcacttcctctgaccacacggttatatataaaaccttaaatgactgtgagctattgggtctagttagcaaacttattggcattcttagcgtctgcttccgatttcttcttcccctaatcaaaatgctgaaggttttctcaccatcaatgttcttttcatttctgctaatgttattcaagccaatgttacgatttggtttgttcgcagcattttcaaacccatgaaaattaggatttccattttcctttacactttctgcattaactctacccacttcatcatcactagtctctgattctaaagcttgcacatctttcttgttttcactgacattattaatgtcctctggcagaacaaaagctgaacttgaattttttcaaacatgacactgtttttgtggctccaaaatgggatcagggattggtttgatgctaggtgctggaagaggaaattcccctggatgacaaccaggccagacagcattgggaccaaaactagatacagctgggttcatattacattgccatgagaactgattgatatgaaatccactgctagtaactggaaaattgttggttggtattgattgaggctgagtgtaaggaggatacatatatggcaacggttgtatcatcatgtttggtgttggtggaggtgatgggtatgtatggtgaggagaggaacacatggggtaactgcaggcagcaagctggcgtagtt

SEQ ID NO: 9 - DNA, 866 bp, Glycine max
cagccgaatgcaatccaacagcggtgaaacttcacctccatttatgaaatcttccagaaactgtacagaatagaagcatctaatttagtaaatacatagggaggaatttatcttgggaacagatccaggacaaaatattagatacatctatacaaaagacaacttaaaattaaggaattggtttcttatcaacactgaaatatcaaaggaaaaaaatggtatcaagcattacggaccatcatacatcagcagaaataaccataacaattaaattaatgcaatgcattttcaggtctatgtttttacaaagtcggctgaacaagcaaaagaaagatgctccatagtaatttcacccaaaagatagcataaataatcagggtaaatatgcaatgtaccagctaaagtcaaaggagaacaataccttggcaggatcatgtgtattatttctcatgaaaaggggggaagtccaaaaacattacaatggaggaaataacgaacatgatgaaccaagaaaacagatcaaagccaacaatcatgactggataatatggcatgcatatattgttttagtttccaataattatcaaaattttgaagcacaacagaaaaatatgcatctatcactgatgccaaaacgaacacaatggaaaaagaaaagcaaataaaaatcagttgaagaggggaattaacaaccttggtatgaggccaaagttgatcaggagatacatgcatagtgcaaccctctttaccagactcccattcaacaacaaaacgagcaaggacacctgaaccaaagctaaaccacaagctcatcagtccaactgcctcaattctaaacgcccttctcatctgttcagttagtttgt

SEQ ID NO: 10 - DNA, 838 bp, Glycine max
agaaatcaccttgcttagataatatgattgggctatgtctataagcattcaaccaacagatgatgatagaatggtataacttacaggaattattccaagacaaggccctcccatgacaccccaaaatccagcccgaaaatcatacctgtatggggggaaatctgttagttcactatagaagcaatcagcagaaaattaataatttgtttgaaattgcaatataacctttaattctgtaaagggaatctaagcatgaaactcatctttattctgtcagtgaacacatactatatacaattgcaatttaactgaatccactaactacaacagcaatttataggagctctttgatatctcaatctaacatagcatccagtataaatggtacagaagataacagctgcccaagtgatcataaagcatactaaccaataatttccaggctgaattgttccggctagcttttcagccttcttgacaacacgatctgataagggctgcccatttacagtaacactgattttgctacgctcatctgtatgattagaccgggagaaatctcggaagcttttcttgataatgtttgcaaaaaatgattcaccacctctgttggatctaggatgatcatgttctcggctagcatctccagaatcctgggaaaccccagtgttagaataatcatggacatccatctctgttgcaagtgctgtctctttcaaagaattttgacgtgctgacatcttgtccacttttgtcttctcctgctctgagcgactactttggtttccctttccaaaccgatttactgcatggttattactagagtaatcaaaatact

SEQ ID NO: 11 - DNA, 523 bp, Glycine max
acccgtctccccaacccagaacccagtcaccggatttggactgcgagagctgccagaaaatggcgtagttccaactgaaattggacacgtggggacgctccaccagatccgacagcttcttctgaagtccctcgtcgctcccaaccgccattaaaacactctcattcggcacggagttcgtcattaggaactccaaagctcgaccacccagaacagccccaagcatggccttgtcctcatcgtcccacaccacgcgccctaaccccgcgtcaaccttcatctcctcaacagaaataaaatacatctattttttcaagcccaaatgcaactttctcagatcttcaagagagagagagagagagagagagagagattgaatatgacgaggctaattaaggtgggaagggggaacaagaaaggaaaactgcaaatgagaatgaaaacaagggtgaattctagcgtgcctaagtaaccttcctgcaccggatgatactttatctgaatcccctgactcctgagaatt

SEQ ID NO: 12 - DNA, 1414 bp, Glycine max
gcataagtatgcgtgagaattcaagcaaataaggaaggaatcgttaagattcttttaagccataccggtttgccgcttagaaccctgagtatctggatactactttgtatcaccaagctgaaatcctttggattctgcttgcagcctcaacctgaactcaagcacccagaaaaacaaaaaaccgctacaactcggttaactatagaatggctaattaaaaatgactaagctgactattaagacataatcagttaccatcaaattgataataacgtgtggttatgataatatccactgtttacacaagaaaaaaaacagagtttactacaatacctataatataatattaataacattatttctataattacaatatcaaaaatggatattacatggaaaaattcatgaggttcttgattagagtgtaagcaacgtcttgcaatacaataaattttaacatgacagtacagtgtaagtatggtattgtttatttttcttccaactagtggttgattccatgcaaactcaacataaattagaatatgacaagccaaccactcaaaagacttgaacttttgttccaataatatatttcttggtgggctcctagttgacttcccaagtctacgcaaaaaatctcctctgcttttaatcttatttctcacttgatcatatcattgcattgccatttctgcaggtcaaatttgcctctaaattactctattggtgacaacactgaaaaattatgcaacataacctgattctcgataccattttcagcatgtcaagtaatgaggttggactttggagaatttcatctattaccacgattttttttggcacgatgccacgatggataaaaaaataggatgattttttcatataatttattttattatcagccctatcctcgaatttttaaagtctcattattaagaaaaatacaagcccaaagaaatagtaattatttataaagtagcattttgcaaataagctgtgacattcatcctacaacttaacccaaataagcaaggagcagaggctttcaagttgattctaactattatttgtaaaaccagaaggtacatgtagagccagattcacggttctcatcctttattttgccatataaaacaagctattgcttatgctctatcttgttggcttgattttgtaatgtgttaccacatacttccattcgatttgctaaatagatagaacaccaatgtcggatatgaatatagacatattgttaacgtgagttcagtaaggtttttggaccacaaccaatccaagggaaaggagatgtaaaaaatactggtccaataaggaattgtgatgctctagtgaaattttcctaagagtagtagttgggtgttaagtaccaaatgcaattagatgcattagcaggtat

SEQ ID NO: 13 - DNA, 502 bp, Glycine max
aggggagccagcaggaggagatagagagtctgtagttggccgttgaatggacttagtggcctcccttggagcaatcattgcttccggactattttcatcttcagaggtactgagagatgaagaatgaaaactcggcatctcccgggacttgttagagttcaaagcaaccagagaaataggttctctgtctactgaatgaaaatcataaccagaataatcatcagatgaaaagttagcaccagtcctgctcacgtgacctcgggaatgtgacatacggctactcacaacctcattagagctattatcacctcttgagggaactccctttgtttctaaattaggagaaataaccagcttcttattgatgacagcaaagctgatttctgaagaacaagccccacattgcaccttttgctggtgatttttcactagaaccaatgcttttttaggcagtagcagcaactcaaagcaattatgacatgagatgaagggcgaaccacca

SEQ ID NO: 14 - DNA, 758 bp, Glycine max
gagtataaacccctccatgtggataaattgctgcataaggaggcccataaggtggcatcataggctggaagaaatagtaagttagcaagttactcatccctgtcatctaggatacatacaaaacatgcaaaaaaaaaaaaaaaaggaaatcatagaacatacaaatgattgcacataggtactactacctgtggtggcccccacatgtacgggtgaggagcgtgaccagaagcaacagctgagttgtagtatggtggcatggtgactcttggcccataatatgcctgaagatatagcagacactttaaatcataaatacagtcactattcactacactagaggccaaatacacttaaaaacttgctgggtgaaccaatttgaaagggataaaattaatagttttataacatcaaatcaatatttcttttaaagattaagcaatccagggacaaagcacatctcatcctagatattaccccaactgattcccaaatccaatagcaattcagtgcataatttcagtctgacgttgattaggggtacatattgatagcaacccacaaactacagaatagaaaatacaatgcaccgcaggacctgcacactaatatttatattcagagagaatgaacagtaattcacaagagggaatataagaagctttcatcagctcattgcttcatccacaaaaaagaaaatggaaatacagtaacagcatttaaataaaaaatagctggtgtcttcccaggaagatgta

SEQ ID NO: 15 - DNA, 666 bp, Glycine max
gagtcgacctgcagcaatccgcggagttcaattcgggtgttaaggatggttgcggctgcttctcactcatcttgcctacttttccaagtcatgcaaccctatgttgcatgaccagaagggagatcaataacagggcttgtcatgaaaatgaaatttgtcctcactatatcagtaatctccactgaatcagcaatgctataatctgcatctcccttagtcttttcactcccacctaacctagaacctaggatagttagaggtttaggatcaagactagcctcagccatgatctcctcgcaacaggtctcggaagcccacggtggtggcggagtgggggcccaccgaggatcggcgtcagcggagacgcagaggacgcagaagtggttgaaggcaactgggcggaggaggaagtggtcacggaagaaggagatgaccttcgatattttgatttatttattttacttaaaaatcttcagaaaatttaaatttaattttaaaacaaatattaaaaggttttgagtcgtcacaaattacatcagatatcatggagcggatagaagaaaatttttatcagatgcaaaatttgaatatatatatcaggcagcatatttaattcaaagaaaattatacatcaagggtttttactctttcattgtgaataatgta

SEQ ID NO: 16 - DNA, 1004 bp, Glycine max
accttatgtgcaagtcgaacctacaacacaacagttcaatgtacaaccaaaatcaatagttaatttgtgaaagtggttgaattaaatgtagtgcagcactagctacaaaattacaaaaccaaatcaaatttatacattatataaataagaaacaaaaagaagcatattgtctcctgaattttagttgggaaaactagtgttacctttcctcactattttgctccaatacatcgaaaagaaaaaggtttctatacataagatataacgtcaaaaaaagggttaaaaaatctcagggggctaatagacaacggtgacaacacagatataacagccttaccactctaatctatgaagtaaatttgataaaacccacatacaaataattttattgtatgctatcaacaaaaactttctaacagaaacagttacccaaagatcattatttgccagatcaacacactagtgcagtgcatgttttgtttttaggtgaaaaggtacttttgaggcaaaagtaattttaccaaaggatgaggaccttgcttttactttcattttacttctatctgttggatttgcattggaatgtgcacaaaacatttctaacttcaatccaaacatgcacttatggttttagttaattttaagaagacagttaacactaaaaagagtctatttaatgcagagttctcagtgtaacaatacaaaatgtatatttatataagaaactgatacttacttgaccagtaaataccacaagaagccgttgctgcaacttggaaatcaactgaggtgaagccaacagagggacaacttgaagccgaagtggaattccaggaaagcttgaagtacatttaatccctgggtacaaaccccctatttggtcctgccagccacctcctgtacccataagttgttctaacactaaaacaagtctagcaacgttctcagtgctatcatccccatcaattacttggagaagccctttcaccactacaggcatgcaa

SEQ ID NO: 17 - DNA, 1069 bp, Glycine max
gattcgccagcttgatgcctgcgacaacaggtgagttctctctcacatatccatctttgttgcttttgactgtcttttcttctccgaaatattcatgcatggctactaccgctcactgggcaaacatggtgttcgatttttttctcttgtcttttctttgatgcacaaattccgagctgttcattctgaggtttggtttggtttgtttaaaagctgacacaaatatgaagttccactagatttaagatttgtttgtatctttgttttaattttctagctatatcaaggatatatatgatattcgtggcaaatatagtagaaaactcgatccaccgtatattggtctcctatcttctccttttgtaccaaaaaaatttatgatcatgatcctaaacacaaaaagcactgccacagctttaacttttactctttaattaccttcttgctcgtgtgatgattattatcaaatatataacaggaaaaagaaaaggattaattattgatttgttattattgttgattcttaaagtttactcattcctcttgttttgactgtcttgtaaattacctccttaatctgttcgcgtaatgaagttaattattaccaagtcatatgcatggtcttgaaattttaatttttatctgtgtgaattcttatcacgtacatgtgaacattacgtaattatagcctcttttggggttggattagctggtgagatccatggaagatataggtccgaattcatgcacaaggccagtgacagagaagaaagcaagaccccaagaacaattgaattgtccaaggtgcagttcaaccaacacaaagttctgttattacaacaactacagcctcacacagccaagatacttctgcaagacttgtagaaggtattggacagaaggagggtctctgagaaacgttcccgttggaggtggctctaggaaaaacaagagggtcacctcatcaaaggttcctgacttgaatccaccaattagcctctcatcagtctcagccatttcttcccaaaaccctaaaatgcaaggagtccatgaccttaatctggctttt

SEQ ID NO: 18 - DNA, 821 bp, Glycine max
  unsure (1)..(821): unsure at all n locations, n = a, t, c, or g
attccataacggtttgcaactcttgaagatcgtgactctggtcgtgtcactcctgcgtatcgcgcctgggagcaacaagattagttgttcctctcatggcttcaatccaccgtttctgctcccattcttcgaaatttcatcggctgcactagtttgtggcttctctaggacaaaatccacaactattttcatgctcatacaaatgcaaaggcacggccacttcgtacagagctgcatcaactcactcttgaaggtcgtactatttctgattatttgactgagattcagaatcttgttgattcttttactgctattggtgatccaatttctatttgcgaacatgttgacattattattgaagaatgtgtaccagaaaactatgagtcctctgtttcgcacatcaataatagatctgaacctctcactattgatgaaatcaaaactgttcttctcggtcatgaggctcagattgacaaattcaggaagaaggcagtggtttcggttaatgttgcttccacatccactgtgtcttctgtgactaatccatctcatgctaattttggaggtttcagaatcagaatcagagtcagtataaaaacagaggacgtagcagtattcagtgttacatctgtcagaagtttggtcatgatgttgccaactgctggcacaggccctcaacttcctatgctctgctccttatcctatgttggcacaatttcccaccatgcctcagctttattccaatttctttggagctgctctgcatttccctcttatctgtttatgcaggctcctgtntctcaacaatgccagcagccact

SEQ ID NO: 19 - DNA, 1395 bp, Glycine max
acttgcctgagagtgttgttgcttctgaacaggctgcatgttcatcacatttgaaagaaactgttggaaaacctactcttgatgcatctcaacccagcccaactgctactcccagagatattgaggcttttggccgatctctaagaccaaacattgttttgaatcataatttctccttgttggatcaagttcaatctgcaagaaacatggagactgatcctagtaatcgggatgtcaagagattgaaagtttctgataatatggtggtggacaaacagctggtagattccaaccatgggcaacagttgtcatatgggtatgataatgtggtcaaagatgggtggtcaggtaataattccatgccatcatcagatcctaatatgctaagcttttcaacaaagccacttgatggacagtacacaaatgcatcttctcaagaggaggttggttatggtaaaaaaattgctcttaatgttgctgacagtaacaaagcagcctctgttaaaagtgattattctctggtaaatcctcaaatggcaccatcatggtttgagcgatatggaacttttaaaaatggtaagatgttgccaatgtacaatgcacagaaaatgactgctgctaagataatggaccagcctttcattgtagcaaaccaattcagatagtttgcgctttcataattcagtagagcaaattcagagtgtcagtgatgctcagctaagtaatgctagtgaaagtccaatgcctgctttagctgcaaataagcatgcagactctcagttatcgacacctgctgttgaacctgacttacttattatgagaccgaagaagcgaaaaagtgccacatctgaactcataccatggcataaagaactgttacagggttctgaaaggcttcgagatatcaggtggttgccaaaactaagtgatttaatgtgcttatttttcggtgttgctattgttggtgtagtaaaagatcccatgtctccagttgatattgtgttgtttcaattgttttgaaagaaaacggtgtgtttccatagtgtcagtatgactattttaatattgttttatgtttatcaatatatcaagtatttgttttcctataacttaaaatttcttactatgtggcagtgtggcagaattagactgggctcaaagtgcaagcagattgattgaaaaggtttgtttataataaaatcagtctacgcatgaatctataattctataatttatgagttcactttactctgtataattataattataggttgaagacagtgtggaggtagttgaagatttgccagcagtggtgaagtcaaaaagaagacttgtcttgtactactcagcttatgcagcaacaacttagtcctcctccagctgcaggcaggcgag

SEQ ID NO: 20 - DNA, 618 bp, Glycine max
atttcttatactcaaatttttggtacctctctttccttcaataaaatttcttcttttatacatgtgtgtgtgtgtgtttggatgttggtaataaatttctgccagaggatttgaagatgaagagtccataagtttgttgattacttgatacaatctaatagagtattttaaccggcccattttttttcttgggctaaagtgatgtaacatctaacaagtgttgaggagataaaacattttcaaggagtttgattgttggatatctagagcaattgtagggttttattgtattcatgatgcttcttaatcattcaaattgtttgtgccttttcatgttatagctttgtgaagaggagttactcaaggaagaagcgcttttagtaaaaaaacaacttatttcctttagttttattaatgacttgtatgcagattggacaacactttagggatggctacttgcataaagaagaatttaagatagtttatgttgctccaatgaaggtatgttgatgcttttgtttttctttacatttctctattcagatttgctttttgttccctgcatttgtgtgccattactcatttctaagtatagattcttgtcctttccaggctttg

SEQ ID NO: 21 - DNA, 1085 bp, Glycine max
tctttcgccagcttgctgcctgcagtaagatccaacctttatggtgggattctatgtcctggataaatctcaaaggtgctatgcctttgagtacaaaacagctgttcatgcagtactcctttttgcaagaagatggtagaaggaataggaggtggcaatactggtggttggctatcacttggtctacttggcagcttcgaaaccgaattctgttctctggagcgacttttgatggaaacaaattggttgaagacgccactttcttaatgtggacttggcttcataatttggagaaggattttactattcatttcaatcactggtccagtaacttcaaacatcattttttgcagtagtaggggtgttttttgggttcatatcacatgtattttcttcccatattttggttcctaatcagacttatgttgcctgattgaggaaccatatttatggtgtctaactctgtatttagtacctctggtacttttctaatatatatatagttttatctttgctgatcaaaaaaaaaaaagtactcatttgtctattcctgtaaagtggcaaggaaataatcagtagctttaaaatcatctgatgtgtggatgtgacacaaattacaacataaatcatgttaaggatatgaaaagtatgtactcattagtctcttccatcaactaagcaaagcaacatggaaatcatctgtagctgagagtgactattaaattgtaagatttggaggtcatggacctcatgtttatagggacagtaaaattatccaattacccaaacctttagacaatctgaaatgcacacataatatggtacaacagttctaaatggggcaggtaagtagcattcattgctcaatatgtctataattcaagaatgaagctttacatttagtgcatatttggacctcagattggcttatttttgctttaagaaaagcttatgatcaaaatgttcaataaaaaacttaaagcttctttttttttttcagtgtaaaatagtcagaaattcagaaccagattgcacttagccagttgtatataaaactatccaatggccataaagaagacagatca

SEQ ID NO: 22 - DNA, 1052 bp, Glycine max
aagttcactcttaactaatgttttttcactgtattccctagctatatttcagactggtgtgtgacagtctttttttgttcatagatattgcggaagcttgaagaacgtggggctgacctagaccgcttgtatgagatggaggaaaaagacattggggcattaattcgttatgcgcctggaggaagggtatgcaacttttactagaatgattttcgaagatttccatcagaggttggttcggatgttgaagaaatgctgattaatgttttcttatcccttcccctttttagttggtcaagcaacacctagggtattttccatcacttcagttatcagcaactgtgagtccaattaccagaactgtgttgaaggtatttcatgatgaagatttttttttccagactgctcagttgacattttttcattgatttcatcacatcaaaaagccttgatacctaattctgcatcaccactcattattttcaggttgatctggtcattacgcctgttttcatttggaaagatcgttttcatggtactgctcaacgttggtggattttggtagaggtgaataaattttcatgtgatgattggtcacattgtaaattccttggtttttgttaaaaactctgatctcttgttataaaaggagaaatttatcaagatgaagagaaagactttcaaagagaaaggaggatgaggaatcctcctaaacaaaggaacaaaacagaaaacaactaggaagaaagagataatcagagaaacaaatcttcccagttgctcgatataactttcagtgaaaatgctaaagaaaccccctttaaagcaaatagatactgagcacctgatcttataccaaatcatgtgacgtgctaaagaaacctcctttaaaaatactagaacagcttgtagcatatgtagcagatttatacaaaaaattagcttctttacttctgtcaaaaccttgaaaaccaatcatcgataattgtttttgagacttaggacacacccaacattaactgaaaatgctgaataagtaatgccagggaggg

SEQ ID NO: 23 - DNA, 855 bp, Glycine max
tagaattacaggtctggagaagtatctgaagactgtagattcggtgcgggattggattctgtttcatatatacttttttaacaacataagttaatttttcatatagttttttatttaattttataaatattttgaataaaaccaaaaatatatgtaagtcgttcgtacataagacgcgttaaacgtcagtacttaataataataatatagtgtaagaaactcaactggggaagtgcataaaaaaataaaagtataaatacaagaaaaatgaactaagaaagtgtgtacttatgtgctaattagcaagatcgttggaacaaaaagccaaattgactggtactttctcgttaatttcttcaattttcattgtttcgttaaatactagtggcatgtccgtcaaaagtcaaaagccacatattgatgaaattgtgttgttagaataattaattaattacttgcagagcaaatctcctccacaatttttctttttttctctacccaagagacttcctttcaactcagatactctttgattctcttcaggaaaacatcaactaattaaaatctaattttgtctttgatactctttgtccgcggaattcaccacccccaccttctcaatttgtttgctttctgctttcttacctcttttttctcagatttcatttggttgatcctttcttcaattcttcttctgggtttgtagttgtttttttatctgacttgtgtttctaaaatccatgaaccgtatgtgatttccagtgtctttttctttttccagattcccagagagaaaaaagaaaaaatccttttgtttgtgtgagactgtaaggatcaattggttgagttctccta

SEQ ID NO: 24 - DNA, 1066 bp, Glycine max
gtatggggcgattcaggaggtggaatctgcaatacaagagcttgaagggaacaatgaggggaatgtaatgttgacagaaactgttggacctgaacacatagccgaggttgttagccgttggactggtatacctgtgacaaggcttggccaaaacgataaagaaaggttgattggtcttgctgacagattgcaccagagagttgtggggcaagaccaagcagttaatgctgttgctgaagctgtgctgagatcaagagctgggcttggaagacctcagcaaccaactggttccttcttgttcttgggtccaactggtgttggcaagactgagctttcaaaggcacttgctgagcaactcttcgatgacgaaaatcaattggtgagaattgacatgtctgaatacatggaacaacactctgtttcgcggttgattggtgcaccaccagggtgtgtggattgacattttcacatttcagtttattgttagttttctgtatgaactacagataactgactcattgtttcgactttcaggtatgttggacatgaagaaggaggtcaactaactgaagctataaggcggaggccttatagtgtggtactctttgatgaagtggaaaaggcacacacatctgtgtttaacactctccttcaagtcttggatgatgggaggttaactgatggccaaggccgtactgtggacttccgaaacactgtcattatcatgacctccaaccttggtgcagagcatctcctcactggactttcaggaaaatcttcaatgcaagtagcccgtgatagagtgatgcaagaggtatgtctcttgacaccatttgtttaatatgtatgacaaaggtctttgtgctgtgttttgacttgtgaccttgtctgttgaatttgttgtaacaggtgaggaggcattttaggccagagttgttgaaccggctcgatgaaattgttgtatttgatcctctttcacacgagcaactaaggaaggtcacaaggttacaaatgaaggacgttgctagtcgtcttgctgagagaggaatagccattggcagtgacc

SEQ ID NO: 25 - DNA, 890 bp, Glycine max
  unsure (1)..(890): unsure at all n locations, n = a, t, c, or g
attatgaagtatgtgacagaattgtgttttcaatatatttctgacaattagggattgcacgggaaaatgaacagcgaccagagaagttatccttaaagaagcaattgaagcggaaatgtctagaacaaaatcccaatcttgtccaaaacccagttatgtgtggatatggagtgggtgctgccagtaaccagcccatggaaatcttgaactgtagccaaccaagtgagaacttgcttatatattaactttctgaggaatacaataaaaaaaaattatttttccttgaagtgatatgttttttcctgtcatacttggtatattggatttaggaagtccctcaaatgtatatcacagcttatattacatgctctcttgtggtaatgcattttcttggtcttaaagattttggccattttagtagataatgtcaaggtagtgagatttgagaattagtgctcttagctgtactcacattagtgttggaacctgttcttcctacttgtttatgtttattgagacaggtaccatggcttgtggcaaggtgatattttctaatggtatataaatataacctataaaaatgtagaccctttatgagcctggaggatcaaagaatggaaaatggaatttggtttattacattcataggggcgaaatgaaatatgctgcatcatgattaccggcagactaaatcccaataaatcatccttttttctganaggaatggtcccgcccagttaggaaaaaactacaggtatcttttgaccgtttgtggaagctctatgagtcggttaaaccgctaactctattcttttatatgcaaggtgtcttctttttcgagtaaacaaatcaaatctcttaaaaaaaagctccggataacttatgtttca

SEQ ID NO: 26 - DNA, 640 bp, Glycine max
aataaatgtcatggactatgatatacctgagcttgttgtttttattcaagaggaacatcagcaatttgtcaaggatacttgcattggtaagggagtgtcaccagaaggcaagtgtatgtcagaagattgtgcagtaaatcatgatccaatgtcatgtcactttgagaatgacttgaacccccgaagagattcaaatctaagaactatggaagcaatgtcaatcaattcaaatgggccagagtttgaatctaaacctcttacccttaaggatgccatggaattttatgattcaagaggtttagtgatggatggtgaagaggattcaggatacaatatttcaattgaccacctcacaaagaagacaataccagagaccattagagaggtgagacactttaatctcatccttgtgcattttacgtctttggacgaggagttaattgttttttagaatattgccactaagacattaatacttatattctaagcaatataaaatatactgtggactcgtcttctcttttagcatggttggtgaaaccccctacatcaatgtacatcttctcttttgtttcatatgcttgattgtatgattgataaaagattgaaacaagacttaataatcatatagggagttacc

SEQ ID NO: 27 - DNA, 21 bp, Artificial sequence
  Description of artificial sequence synthetic primer
caccagtgaagcattggcaat

SEQ ID NO: 28 - DNA, 28 bp, Artificial sequence
  Description of artificial sequence synthetic primer
tggcactacctcacttttgttaaataaa

SEQ ID NO: 29 - DNA, 20 bp, Artificial sequence
  Description of artificial sequence synthetic primer
tcgcgaagagggaactgttt

SEQ ID NO: 30 - DNA, 27 bp, Artificial sequence
  Description of artificial sequence synthetic primer
tgatctcaaatctggtctaaaatccag

SEQ ID NO: 31 - DNA, 22 bp, Artificial sequence
  Description of artificial sequence synthetic primer
cgcaagcaacagagtttcaact

SEQ ID NO: 32 - DNA, 27 bp, Artificial sequence
  Description of artificial sequence synthetic primer
tttgattcttgatggactccctaatta

SEQ ID NO: 33 - DNA, 24 bp, Artificial sequence
  Description of artificial sequence synthetic primer
caggatatattgtttgccatgctt

SEQ ID NO: 34 - DNA, 26 bp, Artificial sequence
  Description of artificial sequence synthetic primer
ccgccaaatataagaacatcttttgt

SEQ ID NO: 35 - DNA, 21 bp, Artificial sequence
  Description of artificial sequence synthetic primer
ctttggattcaacaggtggga

SEQ ID NO: 36 - DNA, 26 bp, Artificial sequence
  Description of artificial sequence synthetic primer
gaggattaatgatgtagccttcaaga

SEQ ID NO: 37 - DNA, 25 bp, Artificial sequence
  Description of artificial sequence synthetic primer
cagaaaaattagcaacaagctcaga

SEQ ID NO: 38 - DNA, 28 bp, Artificial sequence
  Description of artificial sequence synthetic primer
cattgcttactcatttcattttctatcc

SEQ ID NO: 39 - DNA, 27 bp, Artificial sequence
  Description of artificial sequence synthetic primer
aaaagagaattgaatttgtttgattgc

SEQ ID NO: 40 - DNA, 24 bp, Artificial sequence
  Description of artificial sequence synthetic primer
tccccgtgttttcagcattatata

SEQ ID NO: 41 - DNA, 25 bp, Artificial sequence
  Description of artificial sequence synthetic primer
ccattttcctttacactttctgcat

SEQ ID NO: 42 - DNA, 26 bp, Artificial sequence
  Description of artificial sequence synthetic primer
agatgtgcaagctttagaatcagaga

SEQ ID NO: 43 - DNA, 28 bp, Artificial sequence
  Description of artificial sequence synthetic primer
catacatcagcagaaataaccataacaa

SEQ ID NO: 44 - DNA, 25 bp, Artificial sequence
  Description of artificial sequence synthetic primer
cttgttcagccgactttgtaaaaac

SEQ ID NO: 45 - DNA, 29 bp, Artificial sequence
  Description of artificial sequence synthetic primer
gcaatttaactgaatccactaactacaac

SEQ ID NO: 46 - DNA, 25 bp, Artificial sequence
  Description of artificial sequence synthetic primer
cttgggcagctgttatcttctgtac

SEQ ID NO: 47 - DNA, 22 bp, Artificial sequence
  Description of artificial sequence synthetic primer
ccaaccgccattaaaacactct

SEQ ID NO: 48 - DNA, 24 bp, Artificial sequence
  Description of artificial sequence synthetic primer
tcgagctttggagttcctaatgac

SEQ ID NO: 49 - DNA, 28 bp, Artificial sequence
  Description of artificial sequence synthetic primer
aacaccaatgtcggatatgaatatagac

SEQ ID NO: 50 - DNA, 21 bp, Artificial sequence
  Description of artificial sequence synthetic primer
ctttcccttggattggttgtg

SEQ ID NO: 51 - DNA, 20 bp, Artificial sequence
  Description of artificial sequence synthetic primer
cccacattgcaccttttgct

SEQ ID NO: 52 - DNA, 24 bp, Artificial sequence
  Description of artificial sequence synthetic primer
tgagttgctgctactgcctaaaaa

SEQ ID NO: 53 - DNA, 27 bp, Artificial sequence
  Description of artificial sequence synthetic primer
gcataatttcagtctgacgttgattag

SEQ ID NO: 54 - DNA, 25 bp, Artificial sequence
  Description of artificial sequence synthetic primer
gcggtgcattgtattttctattctg

SEQ ID NO: 55 - DNA, 23 bp, Artificial sequence
  Description of artificial sequence synthetic primer
caattcgggtgttaaggatggtt

SEQ ID NO: 56 - DNA, 22 bp, Artificial sequence
  Description of artificial sequence synthetic primer
agggttgcatgacttggaaaag

SEQ ID NO: 57 - DNA, 27 bp, Artificial sequence
  Description of artificial sequence synthetic primer
gttgggaaaactagtgttacctttcct

SEQ ID NO: 58 - DNA, 34 bp, Artificial sequence
  Description of artificial sequence synthetic primer
ttgacgttatatcttatgtatagaaacctttttc

SEQ ID NO: 59 - DNA, 26 bp, Artificial sequence
  Description of artificial sequence synthetic primer
aagtcatatgcatggtcttgaaattt

SEQ ID NO: 60 - DNA, 30 bp, Artificial sequence
  Description of artificial sequence synthetic primer
aagaggctataattacgtaatgttcacatg

SEQ ID NO: 61 - DNA, 19 bp, Artificial sequence
  Description of artificial sequence synthetic primer
cgcctgggagcaacaagat

SEQ ID NO: 62 - DNA, 21 bp, Artificial sequence
  Description of artificial sequence synthetic primer
ttcgaagaatgggagcagaaa

SEQ ID NO: 63 - DNA, 22 bp, Artificial sequence
  Description of artificial sequence synthetic primer
atgggcaacagttgtcatatgg

SEQ ID NO: 64 - DNA, 24 bp, Artificial sequence
  Description of artificial sequence synthetic primer
tgatgatggcatggaattattacc

SEQ ID NO: 65 - DNA, 26 bp, Artificial sequence
  Description of artificial sequence synthetic primer
atttttggtacctctctttccttcaa

SEQ ID NO: 66 - DNA, 26 bp, Artificial sequence
  Description of artificial sequence synthetic primer
ttattaccaacatccaaacacacaca

SEQ ID NO: 67 - DNA, 26 bp, Artificial sequence
  Description of artificial sequence synthetic primer
gtggcaaggaaataatcagtagcttt

SEQ ID NO: 68 - DNA, 31 bp, Artificial sequence
  Description of artificial sequence synthetic primer
atccttaacatgatttatgttgtaatttgtg

SEQ ID NO: 69 - DNA, 24 bp, Artificial sequence
  Description of artificial sequence synthetic primer
ggaggaagggtatgcaacttttac

SEQ ID NO: 70 - DNA, 23 bp, Artificial sequence
  Description of artificial sequence synthetic primer
catttcttcaacatccgaaccaa

SEQ ID NO: 71 - DNA, 27 bp, Artificial sequence
  Description of artificial sequence synthetic primer
cataagacgcgttaaacgtcagtactt

SEQ ID NO: 72 - DNA, 26 bp, Artificial sequence
  Description of artificial sequence synthetic primer
ccaacgatcttgctaattagcacata

SEQ ID NO: 73 - DNA, 20 bp, Artificial sequence
  Description of artificial sequence synthetic primer
cgaggttgttagccgttgga

SEQ ID NO: 74 - DNA, 26 bp, Artificial sequence
  Description of artificial sequence synthetic primer
accaatcaacctttctttatcgtttt

SEQ ID NO: 75 - DNA, 25 bp, Artificial sequence
  Description of artificial sequence synthetic primer
tgtggtaatgcattttcttggtctt

SEQ ID NO: 76 - DNA, 26 bp, Artificial sequence
  Description of artificial sequence synthetic primer
gaacaggttccaacactaatgtgagt

SEQ ID NO: 77 - DNA, 23 bp, Artificial sequence
  Description of artificial sequence synthetic primer
tggaagcaatgtcaatcaattca

SEQ ID NO: 78 - DNA, 22 bp, Artificial sequence
  Description of artificial sequence synthetic primer
tccatggcatccttaagggtaa

SEQ ID NO: 79 - DNA, 15 bp, Artificial sequence
  Description of artificial sequence synthetic probe
acacaagcgcaatta

SEQ ID NO: 80 - DNA, 15 bp, Artificial sequence
  Description of artificial sequence synthetic probe
caagcacaattaaat

SEQ ID NO: 81 - DNA, 18 bp, Artificial sequence
  Description of artificial sequence synthetic probe
cgattttattcaattttt

SEQ ID NO: 82 - DNA, 19 bp, Artificial sequence
  Description of artificial sequence synthetic probe
attgtccgattttattaaa

SEQ ID NO: 83 - DNA, 15 bp, Artificial sequence
  Description of artificial sequence synthetic probe
agagaacggatcaaa

SEQ ID NO: 84 - DNA, 16 bp, Artificial sequence
  Description of artificial sequence synthetic probe
cagagaatggatcaaa

SEQ ID NO: 85 - DNA, 14 bp, Artificial sequence
  Description of artificial sequence synthetic probe
agctccgttatctc

SEQ ID NO: 86 - DNA, 15 bp, Artificial sequence
  Description of artificial sequence synthetic probe
agctccattatctcc

SEQ ID NO: 87 - DNA, 18 bp, Artificial sequence
  Description of artificial sequence synthetic probe
tgatgactatgaataaca

SEQ ID NO: 88 - DNA, 15 bp, Artificial sequence
  Description of artificial sequence synthetic probe
atgaccatgaataac

SEQ ID NO: 89 - DNA, 16 bp, Artificial sequence
  Description of artificial sequence synthetic probe
tcccatagagagactg

SEQ ID NO: 90 - DNA, 17 bp, Artificial sequence
  Description of artificial sequence synthetic probe
tagagacactgaaggtt

SEQ ID NO: 91 - DNA, 17 bp, Artificial sequence
  Description of artificial sequence synthetic probe
aagcaagccctagtata

SEQ ID NO: 92 - DNA, 16 bp, Artificial sequence
  Description of artificial sequence synthetic probe
agcaagccgtagtata

SEQ ID NO: 93 - DNA, 15 bp, Artificial sequence
  Description of artificial sequence synthetic probe
acccaattcatcatc

SEQ ID NO: 94 - DNA, 15 bp, Artificial sequence
  Description of artificial sequence synthetic probe
ctctacccacttcat

SEQ ID NO: 95 - DNA, 15 bp, Artificial sequence
  Description of artificial sequence synthetic probe
tgcattgcattaatt

SEQ ID NO: 96 - DNA, 16 bp, Artificial sequence
  Description of artificial sequence synthetic probe
ctgaaaatgcatcgca

SEQ ID NO: 97 - DNA, 16 bp, Artificial sequence
  Description of artificial sequence synthetic probe
agcatccagtataaat

SEQ ID NO: 98 - DNA, 15 bp, Artificial sequence
  Description of artificial sequence synthetic probe
catccggtataaatg

SEQ ID NO: 99 - DNA, 16 bp, Artificial sequence
  Description of artificial sequence synthetic probe
cattcgacacggagtt

SEQ ID NO: 100 - DNA, 15 bp, Artificial sequence
  Description of artificial sequence synthetic probe
cattcggcacggagt

SEQ ID NO: 101 - DNA, 16 bp, Artificial sequence
  Description of artificial sequence synthetic probe
cgtgagttcagtaagg

SEQ ID NO: 102 - DNA, 18 bp, Artificial sequence
  Description of artificial sequence synthetic probe
acgtgagtttagtaaggt

SEQ ID NO: 103 - DNA, 16 bp, Artificial sequence
  Description of artificial sequence synthetic probe
tttcaccagaaccaat

SEQ ID NO: 104 - DNA, 18 bp, Artificial sequence
  Description of artificial sequence synthetic probe
tttcactagaaccaatgc

SEQ ID NO: 105 - DNA, 15 bp, Artificial sequence
  Description of artificial sequence synthetic probe
tgggttgctatcaat

SEQ ID NO: 106 - DNA, 15 bp, Artificial sequence
  Description of artificial sequence synthetic probe
tgtgggtagctatca

SEQ ID NO: 107 - DNA, 15 bp, Artificial sequence
  Description of artificial sequence synthetic probe
tctcactcatcttgc

SEQ ID NO: 108 - DNA, 15 bp, Artificial sequence
  Description of artificial sequence synthetic probe
tctcagtcatcttgc

SEQ ID NO: 109 - DNA, 18 bp, Artificial sequence
  Description of artificial sequence synthetic probe
cgatgtattggagcaaaa

SEQ ID NO: 110 - DNA, 16 bp, Artificial sequence
  Description of artificial sequence synthetic probe
atgtattgaagcaaaa

SEQ ID NO: 111 - DNA, 14 bp, Artificial sequence
  Description of artificial sequence synthetic probe
tctgtgtgaattct

SEQ ID NO: 112 - DNA, 17 bp, Artificial sequence
  Description of artificial sequence synthetic probe
tttatctgtgtggattc

SEQ ID NO: 113 - DNA, 15 bp, Artificial sequence
  Description of artificial sequence synthetic probe
ctctcatggcttcaa

SEQ ID NO: 114 - DNA, 14 bp, Artificial sequence
  Description of artificial sequence synthetic probe
ctctcgtggcttca

SEQ ID NO: 115 - DNA, 16 bp, Artificial sequence
  Description of artificial sequence synthetic probe
aatgtgatcaaagatg

SEQ ID NO: 116 - DNA, 15 bp, Artificial sequence
  Description of artificial sequence synthetic probe
aatgtggtcaaagat

SEQ ID NO: 117 - DNA, 19 bp, Artificial sequence
  Description of artificial sequence synthetic probe
cacacatgtatataagaag

SEQ ID NO: 118 - DNA, 17 bp, Artificial sequence
  Description of artificial sequence synthetic probe
cacacatgtataaaaga

SEQ ID NO: 119 - DNA, 16 bp, Artificial sequence
  Description of artificial sequence synthetic probe
cacatcctcacatcag

SEQ ID NO: 120 - DNA, 15 bp, Artificial sequence
  Description of artificial sequence synthetic probe
acatccacacatcag

SEQ ID NO: 121 - DNA, 15 bp, Artificial sequence
  Description of artificial sequence synthetic probe
ctctgatggaatcat

SEQ ID NO: 122 - DNA, 14 bp, Artificial sequence
  Description of artificial sequence synthetic probe
tgatggaaatcttc

SEQ ID NO: 123 - DNA, 17 bp, Artificial sequence
  Description of artificial sequence synthetic probe
cttccccatttgagttt

SEQ ID NO: 124 - DNA, 16 bp, Artificial sequence
  Description of artificial sequence synthetic probe
ttccccagttgagttt

SEQ ID NO: 125 - DNA, 15 bp, Artificial sequence
  Description of artificial sequence synthetic probe
ttgtcacgggtatac

SEQ ID NO: 126 - DNA, 16 bp, Artificial sequence
  Description of artificial sequence synthetic probe
tgtcacaggtatacca

SEQ ID NO: 127 - DNA, 15 bp, Artificial sequence
  Description of artificial sequence synthetic probe
tctactcaaatggcc

SEQ ID NO: 128 - DNA, 19 bp, Artificial sequence
  Description of artificial sequence synthetic probe
attatctactaaaatggcc

SEQ ID NO: 129 - DNA, 16 bp, Artificial sequence
  Description of artificial sequence synthetic probe
ccagagtatgaatcta

SEQ ID NO: 130 - DNA, 16 bp, Artificial sequence
  Description of artificial sequence synthetic probe
ccagagtttgaatcta